SEQUENCE LISTING (41 sequences)

SEQ ID NO: 1 - DNA, 21 bp, Artificial
  Synthetic Primer
tggaattctcgggtgccaagg

SEQ ID NO: 2 - DNA, 30 bp, Artificial
  Synthetic Primer
nnnngatcgtcggactgtagaactctgaac

SEQ ID NO: 3 - RNA, 21 nt, Artificial
  Synthetic Primer
ccuuggcacccgagaauucca

SEQ ID NO: 4 - RNA, 26 nt, Artificial
  Synthetic Primer
guucagaguucuacaguccgacgauc

SEQ ID NO: 5 - DNA, 26 bp, Artificial
  Synthetic Primer
ctagcagcctgacatcttgagacttg

SEQ ID NO: 6 - DNA, 22 bp, Artificial
  Synthetic Primer
gccttggcacccgagaattcca

SEQ ID NO: 7 - DNA, 50 bp, Artificial
  Synthetic Primer
aatgatacggcgaccaccgagatctacacgttcagagttctacagtccga

SEQ ID NO: 8 - DNA, 63 bp, Artificial
  Synthetic Primer
caagcagaagacggcatacgagatcgtgatgtgactggagttccttggcacccgagaattcca

SEQ ID NO: 9 - DNA, 28 bp, Artificial
  Synthetic Primer
cgatggatccatggtgagcaagggcgag

SEQ ID NO: 10 - DNA, 28 bp, Artificial
  Synthetic Primer
ggccggatcccttgtacagctcgtccat

SEQ ID NO: 11 - DNA, 30 bp, Artificial
  Synthetic Primer
cgatctcgagacattgaagcagaggttact

SEQ ID NO: 12 - DNA, 29 bp, Artificial
  Synthetic Primer
ggccgaattcatgtttcatttcaccagac

SEQ ID NO: 13 - DNA, 31 bp, Artificial
  Synthetic Primer
ggccgtcgacaaccttgtcataatttttagc

SEQ ID NO: 14 - DNA, 29 bp, Artificial
  Synthetic Primer
cgatgaattcctcagattgttctggtagc

SEQ ID NO: 15 - DNA, 30 bp, Artificial
  Synthetic Primer
cgatgaattcccactctgtgatgaaaatac

SEQ ID NO: 16 - DNA, 30 bp, Artificial
  Synthetic Primer
cgatgaattcaggagaaatacaactggaac

SEQ ID NO: 17 - DNA, 30 bp, Artificial
  Synthetic Primer
cgatgaattctgtaagtttttgccattcta

SEQ ID NO: 18 - DNA, 27 bp, Artificial
  Synthetic Primer
tgaaactgttttattgttgttgcaact

SEQ ID NO: 19 - DNA, 24 bp, Artificial
  Synthetic Primer
atggtattggagtgctttagcctt

SEQ ID NO: 20 - DNA, 30 bp, Artificial; identical to SEQ ID NO: 11
  Synthetic Primer

SEQ ID NO: 21 - DNA, 28 bp, Artificial
  Synthetic Primer
ggccgaattcacaagtaaataaaaggct

SEQ ID NO: 22 - DNA, 29 bp, Artificial
  Synthetic Primer
ggccgaattcatgtttcatttcaccagac

SEQ ID NO: 23 - DNA, 37 bp, Artificial
  Synthetic Primer
cgatctcgagacattgaagcagaggtaactattttat

SEQ ID NO: 24 - DNA, 37 bp, Artificial
  Synthetic Primer
cgatctcgagacattgaagcacaggtaagtattttat

SEQ ID NO: 25 - DNA, 28 bp, Artificial
  Synthetic Primer
cgatgaattcatgtcaggagacggagcc

SEQ ID NO: 26 - DNA, 28 bp, Artificial
  Synthetic Primer
ggccctcgagctaccgaatccgcttctg

SEQ ID NO: 27 - DNA, 28 bp, Artificial
  Synthetic Primer
cgatgaattcatgtcaggagacggagcc

SEQ ID NO: 28 - DNA, 28 bp, Artificial
  Synthetic Primer
ggccctcgagctctgcttcaatgtacag

SEQ ID NO: 29 - DNA, 28 bp, Artificial
  Synthetic Primer
cgatggatccatggtgagcaagggcgag

SEQ ID NO: 30 - DNA, 27 bp, Artificial
  Synthetic Primer
gccgaattccttgtacagctcgtccat

SEQ ID NO: 31 - DNA, 28 bp, Artificial
  Synthetic Primer
cgatgctagcatggcctcctccgaggac

SEQ ID NO: 32 - DNA, 28 bp, Artificial
  Synthetic Primer
ggccctcgagctacaggaacaggtggtg

SEQ ID NO: 33 - DNA, 18 bp, Artificial
  Synthetic Primer
gaggccatgtcaggagac

SEQ ID NO: 34 - DNA, 23 bp, Artificial
  Synthetic Primer
tatcactacagtgaatgctgcaa

SEQ ID NO: 35 - DNA, 18 bp, Artificial
  Synthetic Primer
gaggccatgtcaggagac

SEQ ID NO: 36 - DNA, 23 bp, Artificial
  Synthetic Primer
gctgtaattgccatcagatgcta

SEQ ID NO: 37 - DNA, 18 bp, Artificial
  Synthetic Primer
gaggccatgtcaggagac

SEQ ID NO: 38 - DNA, 21 bp, Artificial
  Synthetic Primer
cataaatcaatgtgcaaaacc

SEQ ID NO: 39 - DNA, 20 bp, Artificial
  Synthetic Primer
cccggctactactacgtgga

SEQ ID NO: 40 - DNA, 24 bp, Artificial
  Synthetic Primer
ctatcactacagtgaatgctgcaa

SEQ ID NO: 41 - DNA, 20 bp, Artificial
  Synthetic Primer
ggacacgctgaacttgttgg

(19) United States
(12) Patent Application Publication
(10) Pub. No.: US 2014/0329700 A1
(43) Pub. Date: Nov. 6, 2014

(54) METHODS OF ISOLATING RNA AND MAPPING OF POLYADENYLATION ISOFORMS

(71) Applicants: Bin Tian, Montvale, NJ (US); Wenting Luo, Boston, MA (US); Zhe Ji, Boston, MA (US); Mainul Hoque, North Arlington, NJ (US)

(72) Inventors: Bin Tian, Montvale, NJ (US); Wenting Luo, Boston, MA (US); Zhe Ji, Boston, MA (US); Mainul Hoque, North Arlington, NJ (US)

(73) Assignee: RUTGERS, THE STATE UNIVERSITY OF NEW JERSEY, New Brunswick, NJ (US)

(21) Appl. No.: 14/240,514

(22) PCT Filed: Aug. 23, 2012
(86) PCT No.: PCT/US12/52122

§ 371 Date: Jul. 24, 2014

Related U.S. Application Data
(60) Provisional application No. 61/526,672, filed on Aug. 23, 2011.
(60) Provisional application No. 61/526,676, filed on Aug. 23, 2011.

(51) Int. Cl. C12Q 1/68

(52) U.S. Cl. CPC C12Q 1/6876; C12Q 1/6806
USPC 506/7; 536/23.1; 435/6.1; 435/6.12; 435/6.19

(57) ABSTRACT

The invention relates to compositions and methods to isolate nucleic acids, and the identification of polyadenylation sites in a gene of interest. In one aspect, the invention provides an oligonucleotide comprising at least one nucleic acid and an affinity moiety, wherein said nucleic acid is 30-60 nucleotides in length and said nucleic acid comprises 1-25 uracil and 5-50 thymine nucleotides.

### CROSS-REFERENCE TO RELATED APPLICATION

This application claims the benefit of U.S. Application Ser. No. 61/526,672 filed Aug. 23, 2011, and U.S. Application Ser. No. 61/526,676 filed Aug. 23, 2011, the disclosures of which are incorporated herein by reference in their entireties.

### STATEMENT REGARDING FEDERALLY FUNDED RESEARCH

This invention was made with government support under Grant GM084089, awarded by the National Institutes of Health. Accordingly, the U.S. Government has certain rights in this invention.

### BACKGROUND OF THE INVENTION

Pre-mRNA cleavage and polyadenylation (polyA) is essential for almost all protein-coding genes in eukaryotes, and is coupled to termination of transcription. The cleavage and polyadenylation site, or polyA site (pA), is defined by surrounding cis elements, including upstream ones, such as UGUA, AAUAAA or its variants (also known as the polyadenylation signal or PAS), and U-rich elements, as well as downstream ones, such as U-rich and GU-rich elements. In mammalian cells, over 20 factors have been shown to be directly involved in polyA. Some proteins form sub-complexes, including the Cleavage and Polyadenylation Specificity Factor (CPSF), containing CPSF160, CPSF100, CPSF73, CPSF30, Fip1L1, and Wdr33; the Cleavage stimulation Factor (CstF), containing CstF77, CstF64, and CstF50; Cleavage Factor I (CFI), containing CFIm68 or CFIm59 and CFIm25; and Cleavage Factor II (CFII), containing Pcf11 and Clp1. CFI and CstF exist as dimers in the polyA complex. A pA in intron 3 of human CstF77 gene, which results in a short mRNA isoform has been previously identified (Gene. 2006 Feb. 1; 366(2):325-34).

Over half of the human mRNA genes have been found to have multiple pAs, leading to mRNA isoforms containing different coding sequences (CDS) and/or variable 3′ untranslated regions (3′UTRs). Alternative cleavage and polyadenylation (APA) plays a significant role in mRNA metabolism by controlling the length of 3′UTR and its encoding cis elements. Dynamic regulation of 3′UTR by APA has been reported in different tissue types, development and cell proliferation/differentiation, cancer cell transformation, and response to extracellular stimuli. By contrast, pAs in introns and upstream exons have not been fully studied at the genomic level. In addition, how APA regulates long non-coding RNAs (IncRNAs), which are increasingly found to play important roles in the cell, is largely unknown.

Identification of pAs typically relies on the cDNA sequence corresponding to the poly(A) tail, which is generated by oligo(dT)-based reverse transcription. However, oligo(dT) can also prime at internal A-rich sequences, which are completely converted to As in the final sequence, becoming indistinguishable from the sequence derived from the real poly(A) tail. This problem, commonly known as the ‘internal priming’ issue, is usually addressed computationally by eliminating putative pAs mapped to genomic A-rich regions. However, this approach not only does not guarantee full elimination of false positives caused by internal priming, but also discards real pAs. In addition, RNA species in the cell can have oligo(A) tails synthesized by noncanonical poly(A) polymerases, such as those involved in exosome-based RNA decay. There is a need to for methods to isolate RNA with a real poly(A) tail, and decrease false positives to identify polyA sites related to cancer cell transformation.

### SUMMARY OF THE INVENTION

In one aspect, the invention provides an oligonucleotide comprising at least one nucleic acid and an affinity moiety, wherein said nucleic acid is 30-60 nucleotides in length and said nucleic acid comprises 1-25 uracil and 5-50 thymine nucleotides.

In a second aspect, the invention provides a method to isolate nucleic acids wherein said method is capable of separating at least one nucleic acid containing a long poly (A) sequence from at least one nucleic acid containing a short poly (A) sequence, said method comprising: obtaining a sample of nucleic acids containing poly (A) sequences; fragmenting said nucleic acids solution to provide a solution of fragmented nucleic acids; reacting said solution of fragmented nucleic acids with the oligonucleotide disclosed herein to provide a solution of nucleic acids annealed to the oligonucleotide and nucleic acids that are not annealed to the oligonucleotide; removing nucleic acids having short poly (A) sequences with a stringent wash to provide a solution of nucleic acids having long poly (A) sequences annealed to the oligonucleotide; contacting said solution of nucleic acids annealed to said oligonucleotide with an enzyme, wherein said enzyme releases nucleic acids from said oligonucleotide; and separating said released nucleic acids to provide a solution of isolated nucleic acids.

In a third aspect, the invention provides a method to detect polyadenylation sites in a gene comprising: obtaining a solution of nucleic acids containing poly(A) sequences; fragmenting said nucleic acids to provide a solution of fragmented nucleic acids; reacting said solution of fragmented nucleic acids with the oligonucleotide of claim 1 to provide a solution of nucleic acids annealed to the oligonucleotide and nucleic acids that are not annealed to the oligonucleotide; removing nucleic acids having short poly (A) sequences with a stringent wash to provide a solution of nucleic acids having long poly (A) sequences annealed to the oligonucleotide; contacting said solution of nucleic acids annealed to said oligonucleotide with an enzyme, wherein said enzyme releases nucleic acids from said oligonucleotide; separating said released nucleic acids to provide a solution of isolated nucleic acids; contacting said solution of purified nucleic acids with a kinase to provide a solution of 5′ phosphorylated nucleic acids; contacting said solution of 5′ phosphorylated nucleic acids with a 3′ adapter, a 5′ adapter, and ligases suitable for ligating said adapters to the 3′ and 5′ ends of the nucleic acids to provide a solution of ligated nucleic acids; contacting said solution with a reverse transcriptase to provide cDNA corresponding to said ligated nucleic acids; amplifying said cDNA corresponding to said ligated nucleic acids by polymerase chain reaction to provide amplified nucleic acids; sequencing said amplified nucleic acids; comparing the sequences of said nucleic acids to the sequence of a reference gene; and determining polyadenylation sites in the gene.

In a fourth aspect, the invention provides a method to determine the differentiation state of a cell comprising: identifying alternative polyadenylation mRNA isoforms of CstF77 from a tissue of interest; determining the ratio of CstF77 short isoforms to CstF77 long isoforms in said tissue, comparing the ratio of CstF77 short isoforms to CstF77 long isoforms in said cell to a standard ratio in a control sample; and wherein if said ratio is greater than a standard ratio in a control sample the state of said cell is a differentiating cell.

In a fifth aspect, the invention provides a method to determine the proliferation state of a cell comprising: identifying alternative polyadenylation mRNA isoforms of CstF77 from a tissue of interest; determining the ratio of CstF77 short isoforms to CstF77 long isoforms in said tissue, comparing the ratio of CstF77 short isoforms to CstF77 long isoforms in said cell to a standard ratio in a control sample; and wherein if said ratio is less than a standard ratio in a control sample the state of said cell is a proliferating cell.

In a sixth aspect, the invention provides a kit comprising the oligonucleotide of as disclosed herein in a single container or separate containers, and instructions for use in a method to detect polyadenylation sites in a gene.

In a seventh aspect, the invention provides a kit comprising a first affinity moiety that binds specifically to a CstF77 short isoform and a second affinity moiety that binds specifically to a CstF77 long isoform in separate containers, and instructions for use in a method to determine the differentiation state of a cell.

In an eighth aspect, the invention provides a computer program product comprising: a computer-readable storage medium; and instructions stored on the computer-readable storage medium that when executed by a computer cause the computer to: receive poly (A) site data; and perform at least one of: (i) mapping poly (A) site data to a genome; (ii) comparing the poly (A) site data in the nucleic acid with a reference nucleic acid; and (iii) identifying a biological marker from the poly (A) site data.

### BRIEF DESCRIPTION OF THE DRAWINGS

FIG. 1(a) illustrates the isolation of nucleic acids; FIG. 1(b) depicts an autoradiograph image that shows the eluted RNA after RNase H digestion, and the A15/A60 ratio indicates the difference in the amount of eluted RNAs containing 15 and 60 As. FIG. 1(c) illustrates the mapping of pAs, and the comparison of the isolated nucleic acid sequences, “reads”, to genomic DNA, and the bottom of FIG. 1(c) illustrates the distribution of three types of reads: 1) reads with 2 As immediately downstream of the last aligned position (LAP), which were used for pA identification and were called polyA site supporting (PASS) reads; 2) reads with <2 As immediately downstream of the LAP, and the LAP is near a pA 24 nt); 3) same as 2) except that the LAP is not near a pA (>24 nt).

FIG. 2 is a schematic of pA types. The full and short names for different pA types are indicated. The number in parenthesis indicates isoform type shown in the graph.

FIG. 3 illustrates the gene structure of human CSTF3, encoding the polyadenylation factor CstF77. Exons are numbered. A polyA site in intron 3 leads to APA isoforms 2 and 3 (isoform 3 has retention of intron 2). Conservation profile is based on vertebrate genomes.

FIG. 4 shows an alignment of vertebrate genomic sequences surrounding the intronic pA of CstF77.

FIG. 5(a) shows a schematic of protein domain structures of CstF77.L and CstF77.S (predicted) and FIG. 5(b) depicts a FACS analysis of HeLa cells transfected with pRinG-77Sin-TT-401 and pRinG-77Sin-AT1690.

FIG. 6 illustrates regulation of intronic polyadenylation of CstF77 in cell differentiation. FIG. 6 (a) depicts expression of CstF77.S (left) and CstF77.L isoforms (right) in C2C12 differentiation. P, proliferating cells; D1, 1 day after differentiation; D4, 4 days after differentiation. FIG. 6(b) shows the CstF77.S/CstF77.L ratio in C2C12 differentiation. FIG. 6(c) shows the P/S ratio of reporter plasmid pRinG77Sin in proliferating and differentiating cells. Different intron sizes were used as indicated. FIG. 6(d) depicts pA usage is lower in differentiating cells compared to proliferating cells.

FIG. 7(a) illustrates a schematic of analysis of the CstF77.S/CstF77.L ratio and global 3′UTR regulation by microarray and RNA-seq data. FIG. 7(b-d) shows the correlation of the CstF77.S/CstF77.L ratio with 3′UTR regulation (RUD) in, FIG. 7(b) C2C12 differentiation, FIG. 7(c) 11 mouse tissues, and FIG. 7(d) 17 human tissues and cell lines. FIG. 7(e) shows a model for regulation of intronic polyA of CstF77 by 3′ end processing and splicing activities.

### DETAILED DESCRIPTION OF THE INVENTION

### 1. Overview

A number of deep sequencing methods for pA analysis exist. However, most of these methods are based on oligo(dT)-priming in reverse transcription, opening the possibility of internal priming. Direct RNA sequencing using the Helicos system (Cell 143(6), 1018 (2010)) does not require reverse transcription, but this method can also be affected by internal A-rich sequences because of using oligo(dT) to fill the poly(A) tail region before sequencing. The key issue with internal priming is that it is impossible to determine whether the unaligned As in reads come from the real poly(A) tail or the oligo(dT) sequence in the primerIn addition, RNA fragments not from genomic A-rich regions can also bind oligo(dT). Surprisingly, these two types of RNA species can account for ˜17% and ˜60% of the total reads generated from CU₅T₄₅ oligo and oligo(dT)₁₀₋₂₅, respectively. Thus, for pA mapping, the method discovered in accordance with the present invention does not use oligo(dT) for priming in reverse transcription, and uses unaligned As in reads for quality control. In the method of the present invention, 3′ region extraction and deep sequencing (3′READS) is not affected by the internal priming issue.

The 3P-seq method (Nature 469 (7328), 97 (2011)) uses splint ligation to ensure that only the RNAs with 3′ terminal As are captured and sequenced, which elegantly addresses the internal priming issue. However, the RNase T1 digestion and multiple steps of ligation and reverse transcription in 3P-seq not only require substantial efforts for optimization of experimental condition but also can introduce noise of various kinds. By contrast, the present invention, 3′READS has fewer steps and is much easier to implement. In addition, 3′READS uses a washing condition that maximally separates long and short A-tailed RNA-species, which can minimize the complication of oligo(A) tails. By contrast, 3P-seq does not address this issue. As such, 3′READS generates 54% more reads usable for pA mapping than 3P-seq.

### 2. Definitions

As used herein, the singular forms “a,” “an” and “the” include plural references unless the content clearly dictates otherwise.

The term “3′READS” is used interchangeably with embodiments of the present invention to isolate nucleic acids, compare nucleic acid sequences, detect and/or map poly (A) sites on another nucleic acid or a gene.

The term “antibody” refers to an immunoglobulin or antigen-binding fragment thereof, and encompasses any such polypeptide comprising an antigen-binding fragment of an antibody. The term includes but is not limited to polyclonal, monoclonal, monospecific, polyspecific, humanized, human, single-chain, single-domain, chimeric, synthetic, recombinant, hybrid, mutated, grafted, and in vitro generated antibodies. The term “antibody” also includes antigen-binding fragments of an antibody. Examples of antigen-binding fragments include, but are not limited to, Fab fragments (consisting of the V_{L}, V_{H}, C_{L} and C_{H}1 domains); Fd fragments (consisting of the V_{H} and C_{H}1 domains); Fv fragments (referring to a dimer of one heavy and one light chain variable domain in tight, non-covalent association); dAb fragments (consisting of a V_{H} domain); single domain fragments (V_{H} domain, V_{L} domain, V_{HH} domain, or V_{NAR} domain); isolated CDR regions; (Fab′)₂ fragments, bivalent fragments (comprising two Fab fragments linked by a disulphide bridge at the hinge region), scFv (referring to a fusion of the V_{L} and V_{H} domains, linked together with a short linker), and other antibody fragments that retain antigen-binding function.

The term “amino acid” refers to natural and/or unnatural or synthetic amino acids, including glycine and both the D and L optical isomers, amino acid analogs (for example norleucine is an analog of leucine) and peptidomimetics.

“Array” as used herein refers to a solid support having a plurality of locations to attach a nucleotide sequence such as a probe or an antibody.

“Animal” includes all vertebrate animals including humans. In particular, the term “vertebrate animal” includes, but not limited to, mammals, humans, canines (e.g., dogs), felines (e.g., cats); equines (e.g., horses), bovines (e.g., cattle), porcine (e.g., pigs), mice, rabbits, goats, as well as in avians. The term “avian” refers to any species or subspecies of the taxonomic class ava, such as, but not limited to, chickens (breeders, broilers and layers), turkeys, ducks, a goose, a quail, pheasants, parrots, finches, hawks, crows and ratites including ostrich, emu and cassowary.

“Attached” or “immobilized’ as used herein to refer to a probe or an antibody and a solid support, refers to the binding between a probe or an antibody and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

A “solid substrate” may be in the form of beads, particles or sheets, a column, an array and may be permeable or impermeable, wherein the surface is coated with a suitable material enabling binding of a target molecule at high affinity. For example, a bead may be coated with strepavidin, and a target molecule bound to biotin will bind to the strepavidin bead with high affinity.

“Probe” as used herein refers to an oligonucleotide capable of binding to a target nucleic acid of complementary sequence through one or more types of chemical bonds, usually through complementary base pairing, usually through hydrogen bond formation. Probes may be directly labeled or indirectly conjugated with an affinity moiety such as with biotin to which a streptavidin complex may later bind. A probe may range in length from 5 nucleotides to a 1000 nucleotides in length, most preferably from 10 to 70 nucleotides in length.

“Biological sample” as used herein means a sample of biological tissue or fluid that comprises polypeptides and/or nucleic acids. Such samples include, but are not limited to, tissue isolated from animals. Biological samples may also include sections of tissues such as biopsy and autopsy samples, frozen sections taken for histologic purposes, blood, plasma, serum, sputum, saliva, stool, tears, mucus, hair, and skin. Biological samples also include explants and primary and/or transformed cell cultures derived from patient tissues. A biological sample may be provided by removing a sample of cells from an animal, but can also be accomplished by using previously isolated cells (e.g., isolated by another person, at another time, and/or for another purpose), or by performing the methods of the invention in vivo.

As used herein, the terms oligonucleotide and chimeric oligonucleotide are used interchangeably.

“Complement” or “complementary” as used herein means Watson-Crick or Hoogsteen base pairing between nucleotides or nucleotide analogs of nucleic acid molecules.

“Conservatively modified variants” applies to both amino acid and nucleic acid sequences. “Amino acid variants” refers to amino acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations”, which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product.

“Identical” or “identity” as used herein in the context of two or more nucleic acids or polypeptide sequences, means that the sequences have a specified percentage of nucleotides or amino acids that are the same over a specified region. The percentage may be calculated by comparing optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces staggered end and the specified region of comparison includes only a single sequence, the residues of single sequence are included in the denominator but not the numerator of the calculation. When comparing DNA and RNA, thymine (T) and uracil (U) are considered equivalent. Identity may be performed manually or by using computer sequence algorithm such as BLAST or BLAST 2.0.

“Label” as used herein may mean a composition detectable by spectroscopic, photochemical, biochemical, immunochemical, chemical, or other physical means. For example, useful labels include radioactive isotopes, fluorescent dyes, electron-dense reagents, enzymes (e.g., as commonly used in an ELISA), biotin, digoxigenin, or haptens, green fluorescent protein, and other entities which can be made detectable. A label may be incorporated into nucleic acids and proteins at any position.

As used herein, the term “linker” refers to a chemical moiety that connects a molecule to another molecule, covalently links separate parts of a molecule or separate molecules. The linker provides spacing between the two molecules or moieties such that they are able to function in their intended manner. Examples of linking groups include peptide linkers, enzyme sensitive peptide linkers/linkers, self-immolative linkers, acid sensitive linkers, multifunctional organic linking agents, bifunctional inorganic crosslinking agents, polymers comprising PEG, PLGA, saccharides, nucleotides, as well as other linkers known in the art. The linker may be stable or degradable/cleavable.

As used herein, the terms poly (A) tail and poly (A) sequence are used interchangeably.

As used herein, the terms “polynucleotide”, “nucleotide sequence” or “nucleic acid” refer to a polymer composed of a multiplicity of nucleotide units (ribonucleotide or deoxyribonucleotide or related structural variants) linked via phosphodiester bonds, including but not limited to, DNA or RNA. The term encompasses sequences that include any of the known base analogs of DNA and RNA. Examples of a nucleic acid include and are not limited to mRNA, miRNA, tRNA, rRNA, snRNA, siRNA, dsRNA, cDNA and DNA/RNA hybrids. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid may be used for the same purpose as a given nucleic acid. Thus, a nucleic acid also encompasses substantially identical nucleic acids and complements thereof. As will also be appreciated by those in the art, a single strand provides a probe for a probe that may hybridize to the target sequence under stringent hybridization conditions. Thus, a nucleic acid also encompasses a probe that hybridizes under stringent hybridization conditions.

As used herein the term “peptide” is used interchangeably with the term “polypeptide”, “protein” and “amino acid sequence”, in its broadest sense refers to a compound of two or more subunit amino acids, amino acid analogs or peptidomimetics.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject. “Substantially complementary” as used herein refers to that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50 or more nucleotides, or that the two sequences hybridize under stringent hybridization conditions.

“Substantially identical” as used herein refers to that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50 or more nucleotides or amino acids, or with respect to nucleic acids, if the first sequence is substantially complementary to the complement of the second sequence.

“Vector” as used herein refers to a nucleic acid sequence containing an origin of replication. A vector may be a plasmid, bacteriophage, bacterial artificial chromosome, yeast artificial chromosome or a virus. A vector may be a DNA or RNA vector. A vector may be either a self-replicating extrachromosomal vector or a vector which integrates into a host genome. The term “expression vector” refers to a nucleic acid assembly containing a promoter which is capable of directing the expression of a sequence or gene of interest in a cell. Vectors typically contain nucleic acid sequences encoding selectable markers for selection of cells that have been transfected by the vector. Generally, “vector construct,” “expression vector,” and “gene transfer vector,” refer to any nucleic acid construct capable of directing the expression of a gene of interest and which can transfer gene sequences to target cells or host cells.

### 3. Oligonucleotide

In one embodiment, the present invention provides an isolated oligonucleotide comprising at least one nucleic acid and an affinity molecule. The nucleic acid may be between 30-60 nucleotides in length and contains uracil and thymine nucleotides, or other molecules similar in structure and affinity that bind to adenine. The uracil can be replaced by other molecules that 1) can base pair with adenine nucleotides, and 2) the paired nucleotides cannot be cleaved by RNase H, for example modified uracil and other derivatives. In certain embodiments, the nucleic acid contains 1-25 uracil and 5-50 thymine nucleotides. In certain embodiments, the uracil nucleotides are contiguous, as well as the thymine nucleotides. In a preferred embodiment, the nucleic acid is 3′-U₅T_{45-5′}, or 3′-U₁₅T_{35-5′}. In certain embodiments, the present invention is a nucleic acid that is substantially complementary and/or substantially identical to U₅T₄₅ or U₁₅T₃₅. The affinity moiety is bound to the nucleic acid, and more than one nucleic acid may be bound to an affinity moiety. In a further embodiment, more than one nucleic acid may be bound to an affinity moiety. The affinity moiety is a molecule that is easily captured, recovered, immobilized or detected. The affinity molecule may be captured by a material attached to a solid support. The oligonuclueotide may also be immobilized to or applied to an array, including a microarray. Examples of an affinity moiety include without limitation, biotin, an antibody, a carbohydrate, a peptide, and a linker. Various types of affinity moieties are known within the skill in the art, as well as the material to enable the affinity moiety to bind to a solid support.

### 4. Methods to Isolate Nucleic Acids

In another embodiment, the present invention provides methods to isolate nucleic acids according to whether the nucleic acid contains a long poly (A) sequence. A long poly (A) sequence is a nucleic acid sequence comprising at least 16 contiguous adenine nucleotides. Samples of nucleic acids containing poly (A) sequences can be obtained from biological samples using any of a number of well-known procedures. In a preferred embodiment, the nucleic acid is RNA, preferably mRNA. Optionally, total RNA can be purified from cell lysates (or other types of samples) using silica-based isolation in an automation-compatible, 96-well format, such as the RNEASY purification platform (QIAGEN, Inc., Valencia, Calif.). RNA can be isolated using solid-phase oligo-dT capture using oligo-dT bound to microbeads or cellulose columns. The sample of nucleic acids that contain poly (A) sequences are then fragmented by methods known in the art, for example with a metal base or metal ion solution such as NaOH or Zn++ solutions, magnesium-sodium periodate fragmentation and fragmentation by .OH radicals, or with ribonuclease(s) such as RNase III. The sample of nucleic acids that contain poly (A) sequences may be fragmented with the Ambion RNA fragmentation kit or NEB RNase III.

Various buffers and solutions are known in the art to fragment RNA. The fragmented nucleic acids containing poly (A) sequences are then isolated using the oligonucleotide of the present invention, i.e., an isolated oligonucleotide comprising at least one nucleic acid and an affinity molecule. The oligonucleotide may be bound to a solid support. In a preferred embodiment, the oligonucleotide is 3′-U₅T₄₅-5′ or 3′-U₁₅T₃₅-5′ conjugated to biotin at its 5′end, and the solid support is beads coated with strepavidin. Nucleic acids with short poly A sequences are removed by stringently washing the solid support while the solid support retains bound nucleic acids containing longer poly (A) sequences. The washing step separates nucleic acids containing long poly (A) sequences from nucleic acids containing short poly (A) sequences, by removing nucleic acids containing short poly (A) sequences from the solid support. This step further enriches the final solution for nucleic acids that contained long poly (A) sequences. In a preferred embodiment the buffer is a low salt buffer, for example 10 mM Tris-HCI pH7.5, 1 mM NaCl, 1 mM EDTA, 10% Formamide or any equivalents thereof. After washing the solid support with a low salt buffer, the solid support and nucleic acids containing poly (A) sequences are then contacted with an enzyme to elute the nucleic acids from the solid support. In a preferred embodiment, the enzyme is RNaseH. RNaseH also removes most of the As of the poly (A) tail, but not As that were base-paired with Us in the oligonucleotide, and thus the eluted nucleic acids correspond to nucleic acids that contained longer poly (A) tails prior to enzymatic digestion. The solution of nucleic acids eluted from the solid support are then purified according to routine methods known in the art.

### 5. Methods to Detect and Map Poly (A) Sites in a Gene

In another embodiment, the present invention also provides methods to detect poly (A) sites in a gene. A purified enriched sample of nucleic acids that contained long poly (A) sequences is obtained as described above. The nucleic acids are then amplified and sequenced according to routine methods in the art. The sequences identified using routine methods in the art are also referred to as reads or READS. These sequences are then compared to a gene or a genome, to identify poly (A) sites. The methods disclosed herein can also be used to compare separate prepared solutions preparations/samples of nucleic acids containing long poly (A) sequences. In a further embodiment, the detected and/or identified poly (A) sites can be recorded in a computer readable form detection data indicating the detection of poly (A) sites in a gene. The present invention further provides methods to identify alternative mRNA polyadenylation isoforms.

In a preferred embodiment, in a solution, the purified sample of enriched nucleic acids that contained long poly (A) sequences are phosphorylated and then the nucleic acids are sequentially ligated to a 3′ adapter and to a 5′ adapter with a ligase. In a preferred embodiment, the 3′-adapter is a 5′-adenylated 3′ adapter. The ligase may be an RNA ligase such as truncated T4 RNA ligase II to ligate the 5′-adenylated 3′ adapter and a T4 RNA Ligase Ito ligate the 5′ adapter. The nucleic acids with the adapters are then reverse transcribed either from the 5′ end (forward sequencing) or the 3′ end (reverse sequencing), and the cDNA is amplified according to known routine methods in the art.

### Bioinformatic Analysis

Candidate loci may be identified by comparison of the isolated nucleic acids with a reference genome using bioinformatic methods known in the art, for example by BLAST comparison with UCSC hg18 (NCBI Build 36) which is a reference assembly for all human DNA sequence. Other databases to compare the indentified poly (A) sites and the corresponding indentified nucleic acid with other nucleic sequences include the Encode Project Consoritum (PLoS Biol 9 (4), e1001046 (2011)), and the exon-exon junction database by Bowtie (B. Langmead, et al., Genome Biol 10 (3), R25 (2009)). Numerous different samples and/or solutions containing nucleic acids that contained long poly (A) sequences may be determined using techniques such as deep sequencing (Shendure and Ji, Nature Biotechnology 26: 1135-1145 (2008)). The methods disclosed herein can further be embodied in a computer readable form such as a computer program product, for example software, by one with ordinary skill in the art.

### Correlation of Location of Poly (A) Sites

Correlation of the location of poly (A) sites in a target nucleic acid sequence provides a useful data set for creating a statistical correlation between the location and strength of poly (A) sites and defined cell characteristics. The amount and/or location of the location of poly (A) sites can be determined. On a molecular level, such correlations can help reveal the strength of the poly (A) site, including the impact of transcription and translation on the function of neighboring sequences, and their related mRNA and peptide isoforms. Such analysis also can identify biomarkers predictive and diagnostic of normal and altered cellular states, e.g. as to whether a cell is in a proliferating state or differentiating state.

### Methods to Determine the State of a Cell

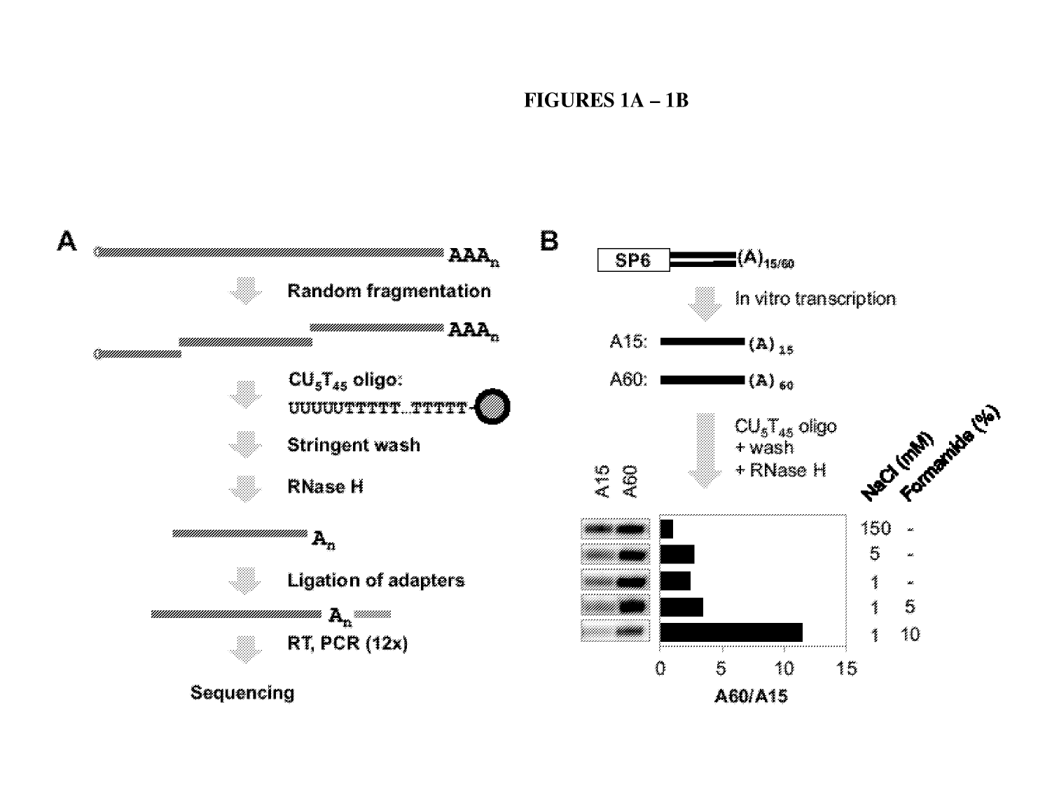

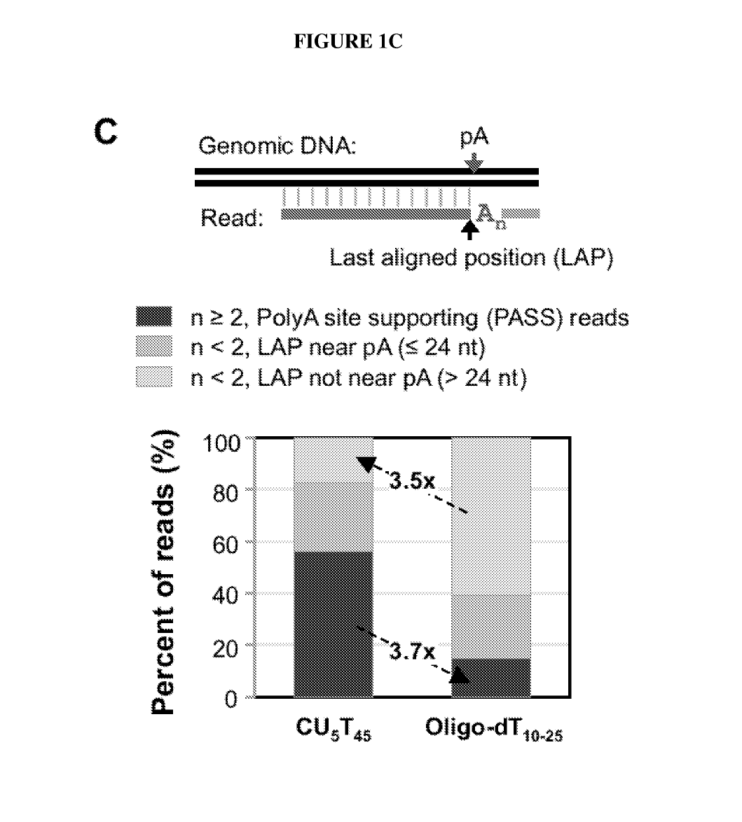

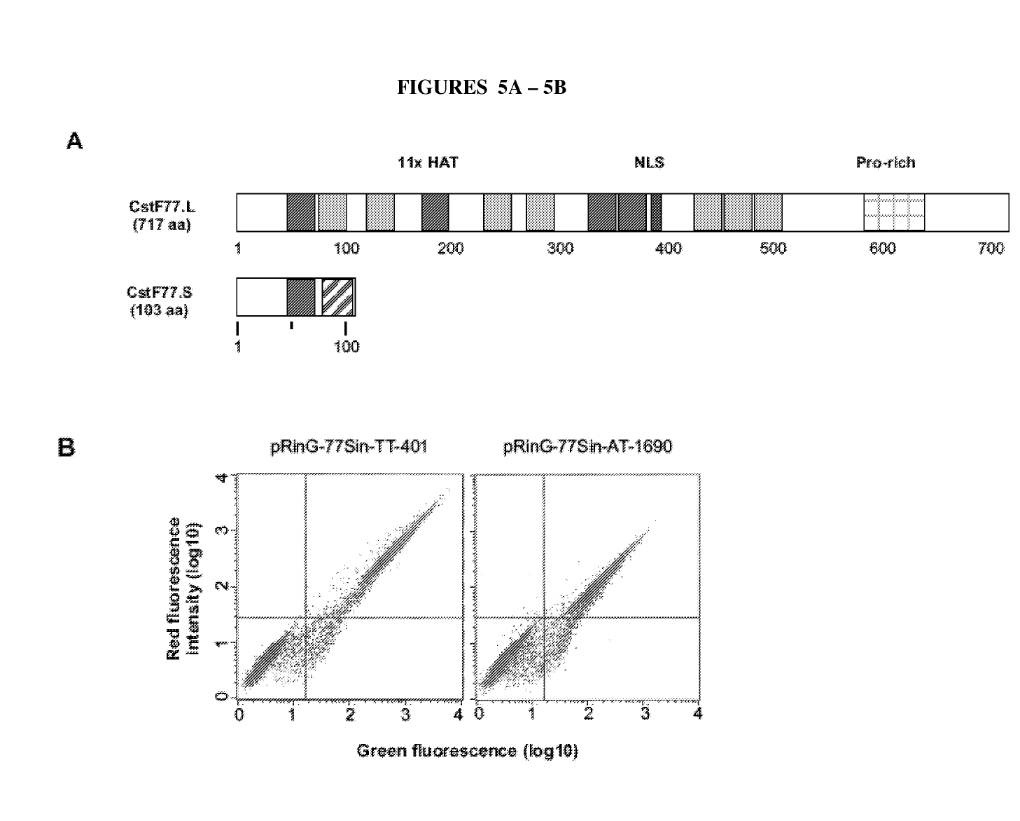

The present invention also provides methods to determine the state of a cell by identifying alternative polyadenylation mRNA isoforms of CstF77 from a cell of interest and determining the ratio of Cstf77 short forms (Cstf3.S) to Cstf77 long isoforms (Cstf3.L) in said cell of interest compared to a standard ratio of Cstf3.S to Cstf3.L in a control sample. The human CstF77 gene (CSTF3) has 21 exons (FIG. 1a), and the conserved intronic pA is located in intron 3. The 5′ portion of the gene before exon 4 accounts for 72% of the gene region. Both introns 1 and 3 are very large, with intron 3 (35.1 kb) being larger than 96.5% of all introns in the human genome and accounting for 47% of the gene, whereas intron 2 is small, below 8% of all introns in the genome (FIG. 1B). Introns 1-3 are highly conserved in size across vertebrates, both in absolute and relative values. (The intronic pA in intron 3 can lead to 2 short CstF77 isoforms (2 and 3, FIG. 1a, and FIG. 5), with or without retention of intron 2, also referred to as CstF77.S collectively. The transcripts with splicing of intron 3 are called a CstF77 long isoform also referred to as CstF77.L. The term “standard ratio” refers to a ratio of Cstf3.S to Cstf3.L in samples of the same type of tissue or cells from subjects who do not have cancer or a cell that is not differentiating; for example, a predetermined standard can be a control level determined based upon ratio of Cstf3.S to Cstf3.L in tissue isolated from subjects who do not have breast cancer, or the ration of Cstf3.S to Cstf3.L in a stem cell from a particular type of tissue. The cell may be a stem cell, an induced pluripotent cell or the cell may be isolated from tissue or from a subject. Routine methods are known in the art to identify mRNA isoforms of CstF77. For example, in C2C12 mouse myoblast cells, if the ratio of Cstf77.S to Cstf77.L is greater than 1 (1 being the arbitrary value of reference), the cell is in a more differentiated state. In other embodiments, if the ratio of Cstf77 short forms to Cstf77 long isoforms is equal to or less than 1 (1 being the arbitrary value of reference), than the cell is in a more differentiating state. One with ordinary skill in the art can determine the standard ratio in normal tissue from a subject compared to cancerous tissue. Based on the correlations and of the ratio of CstF77.S to CstF77.L, the present invention provides assays for CstF77.S to CstF77.L as diagnostic and clinical tools for detecting and diagnosing the proliferation, differentiation, and aberrant cell types that will facilitate study and treatment of a variety of medically relevant states, for example, cancer. In other words, the ratio of Cstf3.S to Cstf3.L in a cell can be used as markers to indicate the state of a cell, such as a cell being in a differentiation state or a proliferation state, such as cancer.

### Kits

The present invention provides kits embodying the methods, compositions, and/or computer materials for the isolation of nucleic acids that contain long poly (A) sequence from nucleic acids that contain short poly (A) sequences, the identification of poly (A) sites in a gene, the identification of mRNA isoforms and the ratio of CstF77.S to CstF77.L in a cell.

Provided herein the kits of the present invention may contain isolated oligonucleotides comprising at least one nucleic acid and an affinity molecule, that anneal to nucleic acids containing long poly (A) sequences with greater affinity compared to nucleic acids with short poly (A) sequences. The oligonucleotide nucleic acid may be between 30-60 nucleotides in length and contains uracil and thymine nucleotides, or other molecules similar in structure and affinity that bind to adenine. In certain embodiments, the nucleic acid contains 1-25 uracil and 5-50 thymine nucleotides. In certain embodiments, the uracil nucleotides are contiguous, as well as the thymine nucleotides. In a preferred embodiment, the nucleic acid is U₅T₄₅ or U₁₅T₃₅. The affinity moiety is bound to the nucleic acid, and more than one nucleic acid may be bound to an affinity moiety. The affinity moiety is a molecule that is easily captured, recovered, immobilized or detected. The affinity molecule may be captured by a material attached to a solid support. The oligonucleotide may also be immobilized to or applied to an array, including a microarray. Examples of an affinity moiety include without limitation, biotin, an antibody, a carbohydrate, a peptide, and a linker.

In a further embodiment, the kit may further contain a nucleic acid described herein together with any or all of the following: assay reagents, buffers, probes and/or primers, and sterile saline or another pharmaceutically acceptable emulsion and suspension base. In addition, the kit may include instructional materials containing directions (e.g., protocols) for the practice of the methods described herein. For example, the kit may be a kit for polymerase chain reaction, amplification, detection, identification, RT-PCR, or quantification of a CstF77 mRNA sequence and related isoforms, CstF77.S and CstF77.L. To that end, the kit may contain a vector, a primer, adapter, and a probe that may further contain a label.

In addition, one or more materials and/or reagents required for preparing a biological sample for gene expression analysis are optionally included in the kit. Furthermore, optionally included in the kits are one or more enzymes suitable for amplifying nucleic acids, including various polymerases (RT, Taq, etc.), one or more deoxynucleotides, and buffers to provide the necessary reaction mixture for amplification.

In a further embodiment, the kits of the present invention may further contain a solid support and reagents. The reagents may be solutions, washing buffers and detection regents. The regents included may be used to bind the oligonucleotide to the solid support. Other reagents include binding buffers, and washing buffers to separate nucleic acids containing long poly (A) sequences from nucleic acids containing short poly (A) sequences, for example a washing buffer may be a low salt buffer that may further contain formanide. Other reagents include enzymes such as an endonuclease, a ligase, an exonucleases, a kinase and RNAse inhibitors to prevent enzymatic degradation of RNA, such as Diethylpyrocarbonate. In certain embodiments, the kit may further contain detection agents that contain a label to identify a nucleic acid sequence of interest.

In a further embodiment, the kits of the invention may contain an oligonucleotide as previously described and a solid support, for example either U₅T₄₅ or U₁₅T₃₅ conjugated to biotin and beads coated with strepavidin.

The kit may be comprised of one or more containers and may also include collection equipment, for example, bottles, bags (such as intravenous fluids bags), vials, syringes, and test tubes. Other components may include needles, diluents and buffers. Usefully, the kit may include at least one container comprising a pharmaceutically-acceptable buffer, such as phosphate-buffered saline, Ringer's solution and dextrose solution.

Optionally, the kits of the invention further include software to expedite the generation, analysis and/or storage of data, and to facilitate access to databases. The software includes logical instructions, instructions sets, or suitable computer programs that can be used in the collection, storage and/or analysis of the data. Comparative and relational analysis of the data is possible using the software provided.

### EXAMPLES

The invention now being generally described, it will be more readily understood by reference to the following examples, which are included merely for purposes of illustration of certain aspects and embodiments of the present invention, and are not intended to limit the invention.

### Methods to Isolate RNA, Detect and Map Poly (A) Sites

### Materials and Methods

Cell Culture and RNA Samples.

Mouse cell lines Tib75, CMT93, B16, F9, and C2C12 were cultured in DMEM with 10% fetal bovine serum (FBS) and NIH3T3, 3T3-L1 and MC3T3-E1 cells were cultured in DMEM with 10% fetal calf serum (FCS). Differentiating C2C12 and 3T3-L1 cells correspond to 4 days and 8 days after initiation of differentiation 10,36,37, respectively. Total RNA from cells was isolated using Trizol (Invitrogen) or the Qiagen RNeasy kit. Mouse whole body tissue RNA sample was purchased from SABiosciences and cell line mix sample was purchased from Agilent. All RNA samples were checked for integrity by Agilent Bioanalyzer using the RNA pico6000 kit (Agilent Technologies). RNA samples with the RNA integrity number (RIN) number above 8.0 were used for subsequent processing.

Plasmids.

Constructs expressing transcripts containing 15 or 60 terminal As, named pALL-A15 and pALL-p60 respectively, were obtained from Dr. Lance Ford (Bioo Scientific). RNAs were made by in vitro transcription using SP6 RNA polymerase.

3′READS.

Total RNA was subjected to 1 round of poly(A) selection using the Poly(A)Purist™ MAG kit (Ambion) according to manufacturer's protocol, followed by fragmentation using Ambion's RNA fragmentation kit at 70° C. for 5 min. Poly(A)-containing RNA fragments were isolated using a chimeric U₅T₄₅ or U₁₅T₄₅ oligonucleotide (CU₅T₄₅ or CU₁₅T₄₅ oligo) (Sigma) which were bound to the MyOne streptavidin C1 beads (Invitrogen) through biotin at its 5′ end. The oligo(dT)₁₀₋₂₅-coated beads were from the Poly(A)Purist MAG kit. Binding of RNA with CU₅T₄₅ or CU₁₅T₄₅ oligo-coated beads was carried out at room temperature for 1 hr in 1× binding buffer (10 mM Tris-HCl pH 7.5, 150 mM NaCl, 1 mM EDTA), followed by washing with a low salt buffer (10 mM Tris-HCl pH7.5, 1 mM NaCl, 1 mM EDTA, 10% Formamide). RNA bound to the CU₅T₄₅ or CU₁₅T₄₅ oligo was digested with RNase H (5U in 50 μl reaction volume) at 37° C. for 1 hr, which also eluted RNA from the beads. Eluted RNA fragments were purified by Phenol:Chloroform extraction and Ethanol precipitation, followed by phosphorylation of the 5′ end with T4 kinase (NEB). Phosphorylated RNA was then purified by the RNeasy kit (Qiagen) and was sequentially ligated to a 5′-adenylated 3′ adapter with the truncated T4 RNA ligase II (Bioo Scientific) and to a 5′ adapter by T4 RNA ligase I (NEB). The resultant RNA was reverse-transcribed to cDNA with Superscript III (Invitrogen), and the cDNA was amplified by 12 cycles of PCR with Phusion high fidelity polymerase (NEB). Adapter sequences were designed so that the RNA fragments can be sequenced from the 5′ end (forward sequencing) or from the 3′ end (reverse sequencing). Adapter sequences and primer sequences are listed in Table 1. cDNA libraries were sequenced on an Illumina Genome Analyzer GAIIx (1×72 nt).

### DATA Analysis

Identification of pA.

For forward sequencing, the reads were aligned to the reference genome (mm9) and exon-exon junction database by Bowtie using the first 25 nt as seed, allowing up to 2 mismatches. Aligned reads were scored from 5′ to 3′ using the scheme: +1 for match and −2 for mismatch. The position in a read with the maximum score was considered as the last aligned position (LAP). The best hit for each read was chosen, and was considered uniquely mapped if its score was greater than the second best hit by at least 5. If a read contained ≧2 non-genomic As immediately after the LAP, the read was considered as a polyA site supporting (PASS) read and the cleavage site is +1 relative to the LAP. For data from reverse sequencing, first the 5′ region of read was trimmed, including the first 4 random nucleotides and subsequent continuous Ts. We then aligned the reads to the reference genome and exon-exon junction database by Bowtie using the first 36 nt, allowing up to 2 mismatches. For uniquely aligned reads, we compared the trimmed Ts with reference genome and exon-exon junction sequences. The reads with at least 2 non-genomic Ts are PASS reads. Since each pA can have multiple cleavage positions in a small window, cleavage positions were merged into pAs: we first clustered together cleavage positions located within 24 nt from one another. If a cluster size was ≦24 nt, the position with the greatest number of PASS reads was used as the representative position for the pA. If a cluster was >24 nt, the first identified cleavage site with the greatest number of PASS reads and re-clustered reads located >24 nt from the position was identified. This process was repeated until all pAs in the cluster were defined. To reduce false positives, a real pA was required to have 1) PASS reads from more than one sample, and 2) ≧2 distinct PASS reads (defined by the number of As and the 4 random Ns) and ≧5% of all PASS reads for the same gene in at least one sample.

Extension of the 3′ End of Genes.

cDNA, EST and directional paired-end RNA-seq data from the ENCODE Project Consortium was used to extend the 3′ ends defined by RefSeq. An extended region is between the 3′ end defined by RefSeq and pAs mapped by 3′READS and is covered by cDNA/EST sequences or RNA-seq reads without a gap greater than 40 nt. It was also required that the 3′UTR extension does not exceed the transcription start site of the downstream gene. For genes located in an intron of another gene, the 3′UTR extension does not go beyond the 3′ SS of the intron.

pAs Flanked by A-Rich Sequences.

A-rich sequence around the pA was defined as ≧6 consecutive As or ≧7 As in a 10 nt window in the −10 to +10 nt region around the pA. pAs located in these regions are typically filtered because they can be derived from internal priming when a primer containing oligo(dT) is used in reverse transcription.

APA Analysis.

The expression level of each APA isoform was indicated by Reads Per Million (RPM) values, which was calculated as the total number of PASS reads normalized to per million total uniquely mapped PASS reads for the sample. For analysis of APA regulation, the Fisher's Exact test was used to examine whether the abundance of an APA isoform compared to that of other isoforms was significantly different between two comparing samples.

lncRNA Genes.

lncRNAs are based on noncoding genes annotated in the RefSeq and Ensemble databases, excluding rRNAs, microRNAs, snoRNAs, snRNAs, and tRNAs, and those overlapping with mRNA genes on the same strand. lncRNAs were required to be longer than 200 nt. Conserved elements were obtained from the UCSC table browser (Euarchontoglires Conserved Elements for mm9) and were mapped to exonic regions of lncRNAs.

Identification of PAS.

To identify PAS, the hexamer with the highest occurrence in the −40 to −1 nt region upstream of all pAs was seleted. Once a hexamer was identified, all associated pAs were removed and the remaining pAs were searched for the next most prominent hexamer. This process was repeated until the top 10 most prominent PAS hexamers were identified.

Cis Element Analysis.

Cis elements in four regions were examiner around the pA, i.e., −100 to −41 nt, −40 to −1 nt, +1 to +40 nt, and +41 to +100 nt. For each region, the difference between observed and expected occurrences (Zₒₑ) for each hexamer were calculated, using

$${Z}_{oe} ( H ) = \frac{{N}_{o} ( H ) - {N}_{e} ( H )}{\sqrt{{v}_{oe} ( H )}} ,$$

where Nₒ(H) is the observed occurrence of hexamer H, Nₑ(H) is the expected occurrence based on the 1^{st}-order Markov Chain model of the region, and vₒₑ(H) is the variance of Nₒ(H)−Nₑ(H) (J. Hu et al., RNA 11 (10), 1485 (2005)).

### 3′ Region Extraction and Deep Sequencing (3′READS).

3′ Region Extraction and Deep Sequencing (3′READS), as Illustrated in FIG. 1A.

After fragmentation of RNA, poly(A)-containing RNA fragments were captured onto magnetic beads coated with a chimeric oligonucleotide (oligo), which contained 45 thymidines (Ts) at the 5′ portion and 5 uridines (Us) at the 3′ portion, dubbed CU₅T₄₅. To optimize washing conditions to enrich RNAs with long poly (A) tails, A15 and A60 were synthesized by in vitro transcription using SP6 RNA polymerase. The sample of RNAs with 60 terminal As were enriched by ˜12-fold as compared to those with 15 As (FIG. 1B). RNase H digestion was used to release RNA from the beads and to remove most of the As of the poly(A) tail. Eluted RNA was ligated to 5′ and 3′ adapters, followed by reverse transcription, PCR amplification, and deep sequencing (see Table 1 for adapter and primer sequences).

TABLE 1
Adapters and primers used in this study.

| | |
|---|---|
| 3′ | Forward sequencing: 5′-rApp/TGGAATTCTCGGGTGCCAAGG/3ddC |
| adapters | (SEQ ID NO: 1) |
| | Reverse sequencing: 5′-rApp/NNNNGATCGTCGGACTGTAGAACTCTGAAC/3ddC¹ |
| | (SEQ ID NO: 2) |
| 5′ | Forward sequencing: 5′-CCUUGGCACCCGAGAAUUCCA (SEQ ID NO: 3) |
| adapters | Reverse sequencing: 5′-GUUCAGAGUUCUACAGUCCGACGAUC (SEQ ID NO: 4) |
| RT | Forward sequencing: 5′-CTAGCAGCCTGACATCTTGAGACTTG (SEQ ID NO: 5) |
| primers | Reverse sequencing: 5′-GCCTTGGCACCCGAGAATTCCA (SEQ ID NO: 6) |
| PCR | 5′-AATGATACGGCGACCACCGAGATCTACACGTTCAGAGTTCTACAGTCCGA (SEQ ID NO: 7) |
| primers | 5′-CAAGCAGAAGACGGCATACGAGAT[CGTGAT]GTGACTGGAGTTCCTTGGCACCCGAGAATTCCA² |
| | (SEQ ID NO: 8) |

¹Ns are random nucleotides used 1) to facilitate separation of clusters on the flow cell by Illumina software (Ts at the beginning of the read can cause problems); and 2) to distinguish different RNA fragments and eliminate redundant reads caused by PCR.
²The nucleotides in bracket are the index used for multiplexing sequencing.

The resulting reads were aligned to the genome, and those with at least 2 non-genomic As at the 3′ end were considered as PolyA Site Supporting (PASS) reads, and were used for polyA site analysis (FIG. 1C).

Using mouse whole body reference RNA, 56% of the reads found generated from 3′READS are PASS reads (FIG. 1C). As expected, the nucleotide profile of the genomic region around the last aligned position (LAP) of these reads is similar to that of pAs that have been reported, indicating that PASS reads are suitable for pA mapping. About 27% of all reads were also aligned near pAs but had no or 1 non-genomic A (FIG. 1C). Presumably, the poly(A) tail sequence of the RNA fragments for these reads has been completely digested by RNase H. The remaining 17% of the reads were distributed along transcripts. About one third of them (6% of total) have the LAP flanked by A-rich sequences, whereas the rest (11% of total) are not aligned to A-rich sequences. Conceivably, the former reads were generated because of binding of RNA with internal A-rich sequences to the CU₅T₄₅ oligo, whereas the latter ones may come from degraded RNAs with oligo(A) tails.

For comparison, a regular oligo(dT) column commonly used for poly(A)+ selection, which contains oligo(dT) 10-25 was used. This column led to far fewer PASS reads (3.7-fold) and more reads mapped to A-rich or other regions (3.5-fold, FIG. 1C), supporting the effectiveness of the method using CU₅T₄₅ or CU₁₅T₄₅ in distinguishing poly(A) tails from internal A-rich sequences. Importantly, since reads containing no additional As after alignment were not used for pA identification, the issue of “internal priming” essentially does not exist. In addition, since the 5 Us in the CU₅T₄₅ or the 15 Us in the CU₁₅T₄₅ oligos can protect some As from digestion by RNase H due to the RNA:RNA base-pairing, the eluted RNAs are more likely to have terminal As than those eluted from oligo(dT)10-25-coated beads (FIG. 1C), making the resultant reads more usable for pA analysis.

To further evaluate the performance of 3′READS, PASS reads mapped to rRNAs, snoRNAs and snRNAs were examined, which are not polyadenylated. Reads mapped to these RNAs would either be due to internal A-rich sequences or the oligo(A) tail produced during their maturation or degradation. The CU₅T₄₅ oligo generated much fewer (5.8-fold) PASS reads mapped to rRNAs/snoRNAs/snRNAs compared to regular oligo(dT)₁₀₋₂₅. 3′READS was compared with several deep sequencing methods recently developed for pA mapping that employed oligo(dT) in reverse transcription, such as PolyA-seq and PAS-seq. 3′READS generated >10-fold fewer reads aligned to rRNAs/snoRNAs/snRNAs, indicating that 3′READS can significant mitigate false positives caused by internal A-rich sequences and oligo(A) tails. The data was compared with those of 3P-seq, which does not use oligo(dT) for priming in reverse transcription. 3′READS gave rise to 54% more usable reads for pA mapping than 3P-seq. This is presumably due to the stringent washing condition and/or fewer sample processing steps used in 3′READS.

### Mapping of pAs in the Mouse Genome.

RNA samples were used from 1) male and female whole bodies, 2) embryos at 11, 15, and 17 days, and 3) over 11 cell lines, yielding ˜42 million PASS reads in total (Table 2).

TABLE 2
Samples used in this study.

| Sample | # of PASS reads |
|---|---|
| Whole body tissue mix (SABiosciences), several male | 3,692,214 |
| and female Balb/c mice, whole bodies without fur. | |
| Cell line mix (Agilent), 11 cell lines | 6,738,355 |
| Cell line mix 2 | 5,972,921 |
| (Tib75, CMT93, B16, F9, MC3T3-E1, N2A, NIH3T3) | |
| Embryos at 11, 15, and 17 days | 7,309,767 |
| Proliferating and differentiating 3T3-L1 cells | 6,631,037 |
| Proliferating and differentiating C2C12 cells | 11,703,170 |
| Total | 42,047,464 |

It was found that 22.6% of the PASS reads were aligned to regions downstream of RefSeq-supported 3′ ends, indicating incomplete gene annotation by the RefSeq database. To address this issue, cDNAs, ESTs and strand-specific paired-end RNA-seq reads from the ENCODE Project Consortium were used to connect the pAs mapped by 3′READS to RefSeq-defined genic regions. This step resulted in extension of the 3′ end for 9,171 genes with the median extension length of 270 nt. The 3′READS data significantly expanded pAs currently annotated for mouse in the PolyA_DB 2 database by more than 2-fold. It was determined that 44% of the pAs are associated with AAUAAA, 15% with AUUAAA, 22% with variants of A[A/U]UAAA, and 19% are not associated with any prominent PAS in the −40 to −1 nt region.

4,818 identified pAs (7.9% of total) are surrounded by genomic A-rich sequences, which would have been filtered out as internal priming candidates if a method employing oligo(dT) in reverse transcription had been used. Except for the A-rich sequence around the cleavage site, these pAs, named A-rich pAs for simplicity, have similar upstream A-rich and downstream U-rich peaks around the cleavage site to regular pAs. This is in contrast to the internal A-rich sequences that led to non-PASS reads. A-rich pAs are more likely to be associated with AAUAAA; their corresponding transcripts are generally more abundant than non-A-rich pAs; and their location distribution in genes is similar to that of non-A-rich pAs. These features further indicate that the A-rich pAs identified correspond to genuine cleavage sites.

Examination of Alternative pAs in the Mouse Genome.

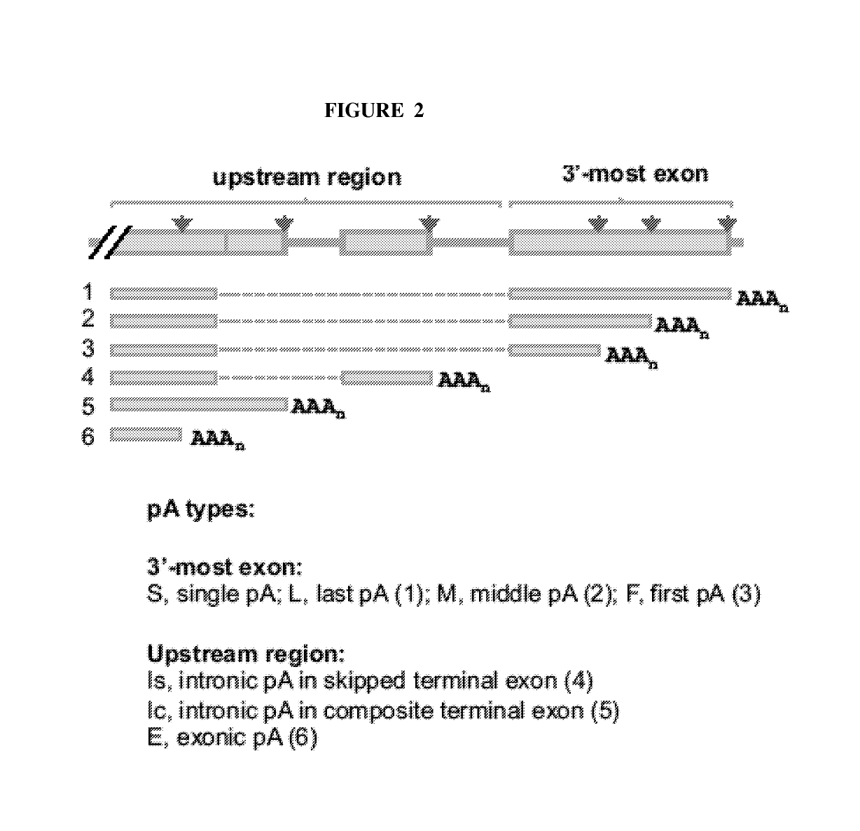

pAs can be located in the 3′-most exon or upstream regions (FIG. 2). pAs in the former group can be further divided into the “single” type when there is only one pA in the 3′-most exon, or the “first”, “middle” and “last” types, according to their relative locations (FIG. 2). pAs in upstream regions can be grouped into the “intronic” type, if there is RefSeq evidence indicating that the pA can be removed by splicing, or the “exonic” type otherwise. For simplicity, intronic and exonic pAs are collectively called I/E pAs. Intronic pAs were further separated into two sub-groups: intronic pAs in skipped terminal exons or composite terminal exons (FIG. 2).

Overall, 17,384 mRNA genes and 1,883 lncRNA genes in the mouse genome were examined. When the relative abundance of an APA isoform was required to be above 5% of all isoforms in at least one sample, 74.7% of mRNA genes and 65.2% of lncRNA genes were found to have APA. On average, there are 3.7 pAs per mRNA gene, and 2.9 pAs per lncRNA gene. Data simulation indicated that the mouse pA collection for mRNA genes is near saturation with the RNA samples used.

mRNA genes were more likely to have alternative pAs in the 3′-most exon, whereas lncRNA genes are more likely to have I/E pAs: 70% of lncRNA genes with APA have I/E pAs compared to 48% for mRNA genes with APA, and was further supported by expression levels of different APA isoforms: for mRNA genes, APA isoforms using 3′-most exon pAs are expressed at much higher levels than those using I/E pAs, whereas the difference between these isoform types is much smaller for lncRNA genes. The PAS pattern for different pA types in lncRNA genes are similar to that in mRNA genes. Overall, the pAs in mRNA and lncRNA genes are surrounded by similar cis elements.

Over 20% of all alternative pAs in mRNA genes are TIE pAs, most of which (>97%) can affect CDS of mRNA. APA regulation in the 3′-most exon on average results in ˜6-fold difference in 3′UTR length for mRNA genes (medians of 301 nt and 1,824 nt for the shortest and longest isoforms, respectively). Therefore, APA can significantly impact the proteome and mRNA metabolism in the cell. To understand the significance of APA for lncRNAs, pA locations relative to conserved elements of lncRNAs were examined, assuming the elements are important for lncRNA functions. It was found that ˜45% of the conserved elements in lncRNAs are downstream of the first I/E pA, and ˜15% are downstream of the first 3′-most exon pA, suggesting that APA can play a significant role in regulation of lncRNA functions.

### Regulation of APA in Development and Differentiation.

C2C12 and 3T3-L1 cells were induced to differentiate, which represent myogenesis and adipogenesis, respectively. In addition, whole embryos at 11 and 15 embryonic days were compared. APA in the 3′-most exon was first examined. Genes having upregulated distal pA isoforms significantly outnumbered those having upregulated proximal pA isoforms in 3T3-L1 differentiation, C2C12 differentiation, and embryonic development (by 5.1-, 2.2-, and 2.1-fold, respectively). In addition, the number of APA events consistently regulated in these processes is significantly greater than that of events oppositely regulated. Distinct APA events in each process can clearly be discerned.

APA of I/E pAs.

All isoforms were grouped together using I/E pAs for each gene and compared its change of abundance with that of isoforms using 3′-most exon pAs, which were also grouped together. The abundance of isoforms using I/E pAs is generally downregulated in development and differentiation: more genes have upregulated 3′-most exon pA isoforms than have upregulated I/E pA isoforms, by 5.6-, 4.0-, and 4.2-fold for 3T3-L1 differentiation, C2C12 differentiation, and embryonic development, respectively. Like APA in the 3′-most exon, both commonly and distinctly regulated APA events in these processes can be identified. pAs are generally upregulated in development and differentiation, regardless of intron/exon locations.

Other Common Features of Isoforms Regulated in Development and Differentiation.

Isoform abundance in the whole body mix and cell line mix samples was first examined. Isoforms upregulated in development and differentiation tend to have higher expression levels in these samples than those downregulated, regardless of their pA locations. This indicates that isoforms with strong pAs are more likely to be upregulated than those with weak pAs. The PAS of pAs of upregulated and downregulated isoforms were examined. Upregulated isoforms are more likely to have pAs associated with AAUAAA than downregulated ones. While 5-mers corresponding to AAUAAA are the most significantly associated with pAs of upregulated isoforms, those related to UGUA upstream elements and UGUG downstream elements were also found statistically significant. Thus, pA strength is a significant parameter in determining APA regulation in development and differentiation.

### CSTF77

### Materials and Methods

Plasmids.

Construction of the pRinG vector and all plasmids derived from pRinG are described in Table 3. See Proc Natl Acad Sci USA 106: 7028-7033 regarding the pRiG vector

TABLE 3
Constructs used for reporter assays

| | |
|---|---|
| pRinG | A fragment containing the open reading frame |
| | (ORF) of EGFP was generated by PCR using |
| | pIRES2-EGFP (BD Biosciences) as template |
| | and primers 5′-CGATGGATCCATGGTGAGCAAGGGCGAG |
| | (SEQ ID NO: 9) and 5′-GGCCGGATCCCTTGTACAGCTCGTCCAT |
| | (SEQ ID NO: 10). The fragment was cut by BamH I and |
| | inserted into the pDsRED-Express-c1 vector |
| | (BD Biosciences) digested with the same enzyme. |
| pRinG-77Sin-5′SS | The genomic sequence including the 5′SS |
| | of exon 3 and pA of intron 3 in human CSTF3 was |
| | amplified by PCR using the genomic DNA from HeLa |
| | cells with primers 5′-CGATCTCGAGACATTGAAGCAGAGGTTACT |
| | (SEQ ID NO: 11) and 5′-GGCCGAATTCATGTTTCATTTCACCAGAC |
| | (SEQ ID NO: 12). This sequence contains 24 nt of |
| | exon 3 and 212 nt downstream of the AUUAAA PAS of the |
| | intronic pA. The PCR product was cut by Xho I and |
| | EcoR I and then inserted into the pRinG vector |
| | digested with same enzymes. |
| pRinG-77Sin-401 | The fragments containing different 3′ regions |
| pRinG-77Sin-831 | of intron 3 and a portion of exon 4 (27 nt) were |
| pRinG-77Sin-1690 | generated from the same genomic DNA using a common |
| pRinG-77Sin-2378 | reverse primer 5′-GGCCGTCGACAACCTTGTCATAATTTTTAGC |
| | (SEQ ID NO: 13), and specific forward primers: 5′- |
| | CGATGAATTCCTCAGATTGTTCTGGTAGC (SEQ ID NO: 14) for |
| | pRinG-77Sin-401, 5′-CGATGAATTCCCACTCTGTGATGAAAATAC |
| | (SEQ ID NO: 15) for pRinG-77Sin-831, 5′- |
| | CGATGAATTCAGGAGAAATACAACTGGAAC (SEQ ID NO: 16) |
| | for pRinG-77Sin-1690 and 5′- |
| | CGATGAATTCTGTAAGTTTTTGCCATTCTA (SEQ ID NO: 17) for |
| | pRinG-77Sin-2378. The PCR products were cut by EcoR |
| | I and Sal I and then inserted into pRinG-77Sin-5′SS |
| | digested with same enzymes. |
| pRinG-77Sin-401-AT | The AUUAAA PAS was mutated by the Phusion ™ |
| pRinG-77Sin-831-AT | Site-Directed Mutagenesis Kit (NEB) according |
| pRinG-77Sin-1690-AT | manufacturer's protocol using primers |
| pRinG-77Sin-2378-AT | 5′-TGAAACTGTTTTATTGTTGTTGCAACT (SEQ ID NO: 18) |
| | and 5′-ATGGTATTGGAGTGCTTTAGCCTT (SEQ ID NO: 19). |
| pRinG-77Sin-401-noGU | For pA mutants without the downstream GU-rich |
| pRinG-77Sin-831-noGU | element, we first generated a PCR product using |
| pRinG-77Sin-1690-noGU | pRinG-77Sin-401 as template and primers |
| pRinG-77Sin-2378-noGU | 5′-CGATCTCGAGACATTGAAGCAGAGGTTACT (SEQ ID NO: 20) |
| | and 5′-GGCCGAATTCACAAG TAAATAAAAGGCT (SEQ ID NO: 21), |
| | and used it to replace the sequence between 5′SS |
| | and pA in the template construct using Xho I and EcoR I. |
| | The inserted sequence lacks a 156 nt region downstream of the pA site. |
| | The intronic sequence containing 3′SS was replaced with |
| | corresponding sequences containing different fragments |
| | (831nt, 1,690 nt, and 2,378 nt) by compatible restriction enzymes. |
| pRinG-77Sin-401-AT-noGU | As for pRinG-77Sin-401/831/1690/2378-noGU, except |
| pRinG-77Sin-831-AT-noGU | that pRinG-77Sin-401-AT was used as template for cloning. |
| pRinG-77Sin-1690-AT-noGU | |
| pRinG-77Sin-2378-AT-noGU | |
| pRinG-77Sin-401-AT-5′SSMT1 | For constructs containing 5′SS mutations, we |
| pRinG-77Sin-831-AT-5′SSMT1 | first introduced mutations to 5′SS by PCR using |
| pRinG-77Sin-1690-AT-5′SSMT1 | pRinG-77Sin-401-AT as template, a common reverse primer |
| pRinG-77Sin-2378-AT-5′SSMT1 | 5′-GGCCGAATTCATGTTTCATTTCACCAGAC (SEQ ID NO: 22), |
| pRinG-77Sin-401-AT-5′SSM2 | and 2 different forward primers, |
| pRinG-77Sin-831-AT-5′SSM2 | 5′-CGATCTCGAGACATTGAAGCAGAGGTAACTATTTTAT |
| pRinG-77Sin-1690-AT-5′SSM2 | (SEQ ID NO: 23) for mutant 1 (MT1) 1 and |
| pRinG-77Sin-2378-AT-5′SSM2 | 5′CGATCTCGAGACATTGAAGCACAGGTAAGTATTTTAT (SEQ ID NO: 24) |
| | for mutant 2 (MT2). PCR products were cut by Xho I and |
| | EcoR I and were used to replace corresponding sequences |
| | containing the wild type 5′SS in different vectors. |
| | The intronic sequence containing 3′SS was replaced |
| | with corresponding sequences containing different fragments |
| | (831 nt, 1,690 nt, and 2,378 nt) by compatible restriction enzymes. |

For pCMV-CstF77, the open reading frame (ORF) of human CstF77 was obtained from the IMAGE clone 5223351 (Invitrogen) by PCR using primers 5′-cgatgaattcatgtc aggagacggagcc (SEQ ID NO: 25) and 5′-ggccctcgagCTACCGAATCCGCTTCTG (SEQ ID NO: 26). The fragment was cut by EcoR I and Xho I, and then inserted into the pcDNA3.1/His C vector (Invitrogen) digested with the same enzymes. For pCMV-CstF77S, a fragment containing the coding region of exons 1-3 of CstF77 was generated by PCR using pCMV-CstF77 and the primers 5′-cgatgaattcatgtcaggagacggagcc (SEQ ID NO: 27) and 5′-ggccctcgag CTCTGCTTCAATGTACAG (SEQ ID NO: 28), and the fragment was used to replace the CstF77 ORF by EcoRI and XhoI. For pCMV-77L-EGFP and pCMV-77S-EGFP, we obtained the ORF of EGFP from pIRES2-EGFP (BD Biosciences) using primers primers 5′-cgatggatccATGGTGAGCAAGGGCGAG (SEQ ID NO: 29) and 5′-GCCGAATTCCTTGTACAGCTCGTCCAT (SEQ ID NO: 30). The PCR products were digested with BamH I and EcoR I, and were inserted into the pCMV-77L or pCMV-77S vectors that were digested with the same enzymes.

Cell Culture, Differentiation and Transfection.

HeLa cells and C2C12 cells were maintained in Dulbecco's Modified Eagles Medium (DMEM) supplemented with 10% fetal bovine serum (FBS). Differentiation of C2C12 cells was induced by switching cell media to DMEM+2% horse serum (Sigma) when cells were ˜100% confluent. All media were also supplemented with 100 units/ml penicillin and 100 μg/ml streptomycin. Transfection was carried out by Lipofectamine™ 2000 (Invitrogen) or jetPEI™ (polyplus) according to manufacturer's recommendations.

FACS and Immunoblot.

For fluorescent activated cell sorting (FACS) analysis, cells were released from culture dishes by Trypsin-EDTA 24 h after transfection and green and red fluorescence were read at 530 nm and 585 nm, respectively, in the BD FACScalibur system (BD Biosciences). For immunoblot, the RIPA buffer (1% NP-40, 0.1% SDS, 50 mM Tris-HCl pH 7.4, 150 mM NaCl, 0.5% Sodium Deoxycholate, and 1 mM EDTA) was used to extract proteins from the cell. Proteins were resolved by SDS-PAGE, followed by immunoblotting using anti-RFP antibody (BD clonetech), anti-Xpress antibody (Invitrogen), anti-Omni tag antibody (Santa Cruz), or anti-CstF77 antibody (Santa Cruz).

Northern Blot and RT-qPCR.

Total cellular RNA was extracted using Trizol (Invitrogen) according to manufacturer's protocol. mRNA was reverse-transcribed using the oligo-dT primer (Promega). For Northern blot, total RNA was run in a 1.2% denaturing agarose gel, and was transferred to nylon membrane overnight. RNA expression was detected by hybridization with a radioactively labeled probe for the RFP sequence. The probe was made by PCR using pD sRED-Expres s-c1 as template and primers 5′-CGATGCTAGCATGGCCTCCTCCGAGGAC (SEQ ID NO: 31) and 5′-GGCCCTCGAGCTACAGGAACAG GTGGTG (SEQ ID NO: 32) with α-³²P-dCTP. RT-qPCR was carried out with Syber-Green I as dye. RT-qPCR primers used for human and mouse CstF77.S and CstF.L are as follows: Human CstF77.S: 5′-GAGGCCATGTCAGGAGAC (SEQ ID NO: 33) and 5′-TATCACTACAGTGAATGCTGCAA (SEQ ID NO: 34), Mouse CstF77.S: 5′-GAGGCCATGTCAGGAGAC (SEQ ID NO: 35) and 5′-GCTGTAATTGCCATCAGATGCTA (SEQ ID NO: 36), Human and mouse CstF77.L: 5′-GAGGCCATGTCAGGAGAC (SEQ ID NO: 37) and 5′-CATAAATCAATGTGCAAAACC (SEQ ID NO: 38). The following primers were also used for detection: F1 (5′-CCCGGCTACTACTACGTGGA-3′ (SEQ ID NO: 39)), R1 (5′-CTATCACTACAGTGAATGCTGCAA-3′ (SEQ ID NO: 40)), R2 (5′-GGACACGCTGAACTTGTTGG-3′ (SEQ ID NO: 41)).

Analysis of DNA Microarray and RNA-Seq Data.

To calculate the global 3′UTR length (RUD) score, we used exon array and RNA-seq datasets (Proc Natl Acad Sci USA 106: 7028-7033 (2009). Exon array data were first normalized by the Robust Multichip Average (RMA) method. Expressed genes were selected by the Detection Above Background (DABG) method. The RUD score was based on the ratio of average probeset intensity in aUTR to that in cUTR, as previously described (Ji et al., 2009). For RNA-seq data, the RUD score was based on the ratio of read density in aUTR to that in cUTR, as described previously (Mol Syst Biol 7: 534 (2011)). For both analyses, aUTRs and cUTRs were defined by PolyA_DB 2 (Nucleic Acids Res 35: D165-168 (2007)).

Analysis of Introns.

5′SS and 3′SS were analyzed (Genome Res 17: 156-165 (2007)). Briefly, All GT-AT type introns of human RefSeq genes were usedto build Position Specific Scoring Matrices (PSSMs) for 5′SS and 3′SS. For 5′SS, −3 to +6 nt surrounding the 5′SS were used with 3 nt in the exon and 6 nt in the intron; for 3′SS, −22 to +2 nt surrounding the 3′SS were used, with 22 nt in the intron and 2 nt in the exon. The maximum entropy scores were calculated by MaxEnt (J Comput Biol 11: 377-394 (2004)). 5′SS sequences were also scored by their ability to hybridize with U1 SnRNA. The sequence 5′-ACTTACCTG of U1 SnRNA was used to form duplex structures with 5′SS sequences using the RNAduplex function of ViennaRNA(Nucleic Acids Research 31: 3429-3431 (2003)). For intron density map, all the RefSeq-supported introns were used as the observed set, and created an expected set using randomized pairs of intron size and splice site. The introns were divided into 20 fractions based on intron size and splice site strength, respectively, and distributed in a 20×20 grid. For each cell in the grid, the ratio of the number of introns in the observed set to that in the expected set was calculated and represented by color in a heatmap.

### Conserved, Unique Splicing Features Surrounding the Intronic pA of the Human CstF77 Gene.

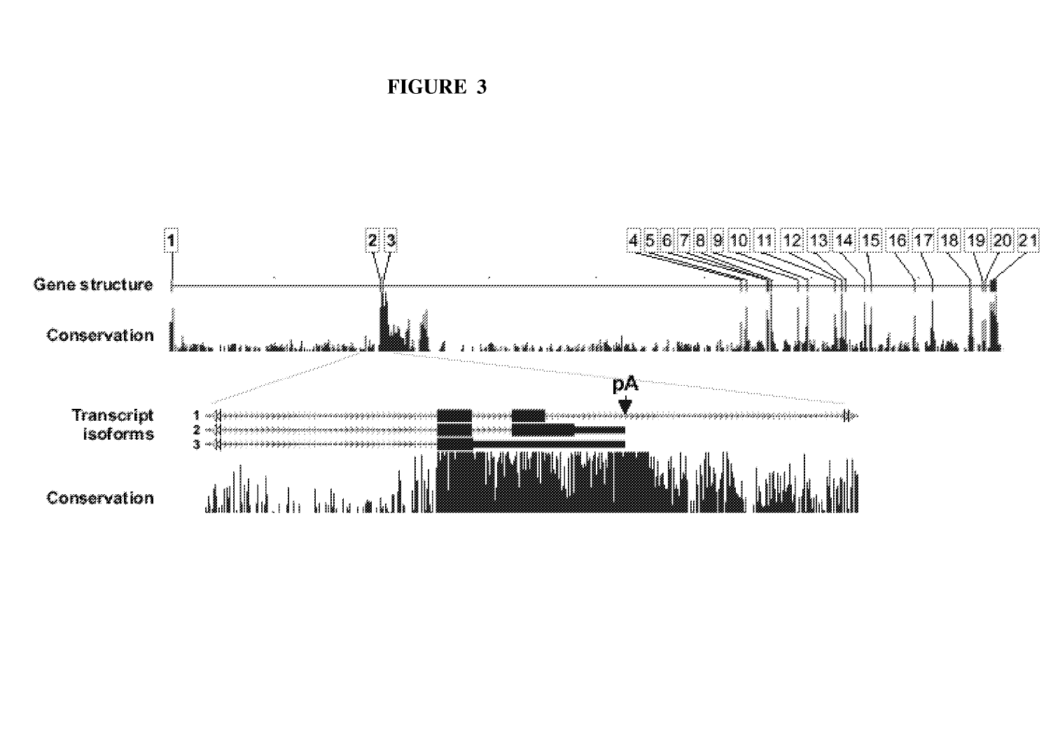

The human CstF77 gene (CSTF3) has 21 exons (FIG. 3), and the conserved intronic pA is located in intron 3. Remarkably, the 5′ portion of the gene before exon 4 accounts for 72% of the gene region. Both introns 1 and 3 are very large, with intron 3 (35.1 kb) being larger than 96.5% of all introns in the human genome and accounting for 47% of the gene, whereas intron 2 is small, below 8% of all introns in the genome. In addition, introns 1-3 are highly conserved in size across vertebrates, both in absolute and relative values, suggesting functional relevance. The intronic pA in intron 3 can lead to 2 short isoforms (2 and 3 in FIG. 3), with or without retention of intron 2, these two short isoforms are referred to as CstF77.S collectively. The transcripts with splicing of intron 3 are referred to as CstF77.L.

It was concluded that the 5′SS of intron 3 is weak based on several measurements, including using position-specific scoring matrix (PSSM) derived from all human introns, maximum entropy (ME) score which takes into account co-variation of nucleotides (Comput Biol 11: 377-394 (2004)), and free energy of base pairing with the U1 snRNA. It is at the 0.7^{th}-percentile of all introns in the human genome. By contrast, the 3′SS region is quite strong, at the 94.3^{th}-percentile of all introns. Importantly, both the 5′SS and 3′SS regions are highly conserved across vertebrates, compared to those regions of other introns. Notably, intron 2 also has a relatively weak 5′SS, which may be responsible for intron retention of some CstF77.S mRNAs.

Since splicing in higher species is typically carried out according to the exon-definition model, the 5′SS and 3′SS regions of upstream and downstream exons were examined. Intron density maps were used to simultaneously interrogate intron size and 5′SS or 3′SS strength. In general, large introns in the human genome are flanked by strong 5′SS and 3′SS in both upstream and downstream exons. This principle holds for the upstream 3′SS and downstream 5′SS and 3′SS of intron 3 of CSTF3, but not for the upstream 5′SS. Therefore, the combination of large intron size and weak 5′SS of intron 3 is unique in the genome.

### Perturbations of Splicing and Polyadenylation Parameters Impact Intronic Polyadenylation.

Reporter constructs were generated to examine the significance of various features surrounding the intronic pA of CSTF3, (called pRinG) containing the 5′ and 3′ regions of intron 3 and partial sequences from exons 3 and 4. The 5′ region contained the intronic pA. Depending upon the usage of the intronic pA, a short isoform (isoform P) containing RFP or a long isoform (isoform S) containing RFP and EGFP could be expressed. To understand the impact of intron size, 3′ regions of intron 3 was cloned with various sizes. As the insert size increased the amount of intronic pA product went up. There was a linear increase of intronic pA usage, based on log₂(isoform P/isoform S), for insert sizes from 401-1690 nt. However, no further increase could be observed for the 2,378 nt insert. To confirm that the regulation of intronic pA usage is due to change of intron size rather than the distance between the intronic pA and the SV40 pA at the 3′ end of the reporter gene, the region after 3′SS was expanded by adding another EGFP sequence. However, no significant difference could be discerned. By contrast, a linear decrease of intronic pA usage was observed when the distance between 5′SS and pA was expanded by random sequences. Taken together, these data indicate that intron size is important for the usage of intronic pA, indicating kinetic competition between polyA and splicing.

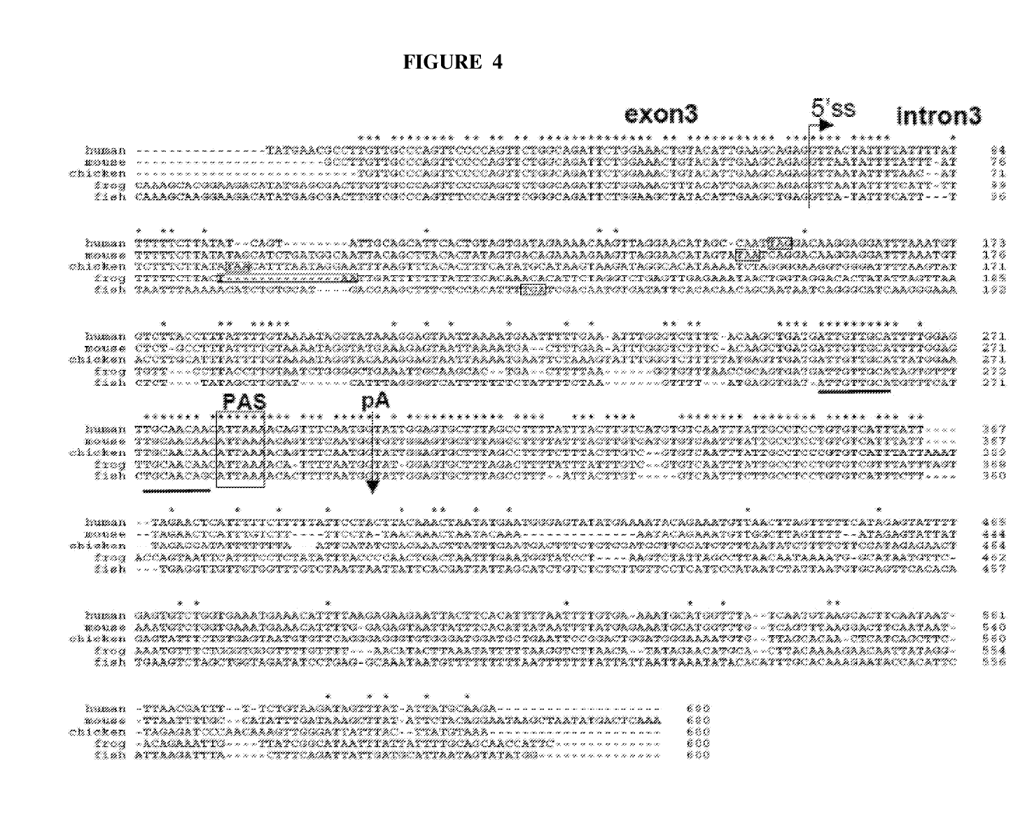

The region surrounding the intronic pA is highly conserved across vertebrates, including the upstream AUUAAA element and downstream U-rich and UG-rich elements (FIG. 4). Since AUUAAA has a lower 3′ end processing activity than the canonical AAUAAA element, to determine whether the intronic pA of CstF77 has medium strength, AUUAAA was mutated to AAUAAA, and/or deleted the downstream GU-rich element. It was found that using AAUAAA led to ˜2 fold increase in pA usage, and deletion of the GU-rich element led to ˜10 fold decrease in pA usage. This analysis confirms that the pA strength is at a suboptimal level. The slope of the intronic pA usage curve, based on log₂(P/S) vs. distance between pA and 3′SS, for constructs with the GU-rich element appeared different compared with those without, suggesting that contribution of GU-rich element to pA strength may be different than that of PAS.

The importance of strengths of 5′SS and 3′SS was examined. The 5′SS sequence was mutated to stronger sequences (mutants 1 and 2) based on the strength score calculated by ME. Mutants 1 and 2 would be at the 51.9^{th}- and 95.5^{th}-percentile in the human genome with respect to the ME score. As shown in FIG. 2D, strengthening 5′SS dramatically inhibited intronic pA usage: ˜90% decrease for mutant 1 and no detectable intronic pA usage for mutant 2. Thus, 5′SS strength is very critical for the intronic polyA. 3′SS was also mutated, weakening its strength to the 3.8^{th}-percentile based on the ME score. However, only a minor effect (˜30%) can be discerned. Thus, compared to 3′SS, 5′SS plays a dominant role in regulation of intronic pA.

The CstF77.S mRNA does not Lead to a Detectable Protein

The CstF77.S mRNA would encode a protein of 103 amino acids (aa), containing the N-terminal region of CstF77 and some aa from the intronic region (FIG. 5a). However, the CstF77.S protein product could not be detected using various antibodies against the N-terminal region of CstF77. It was observed using FACS analysis of HeLa cells transfected with various pRinG constructs, the ratio of red to green fluorescence intensities is constant across all constructs, and the constructs generating more intronic isoforms have both decreased red and green fluorescence intensities (examples shown in FIG. 5b). An immunoblot analysis using an antibody against RFP confirmed that the CstF77.S mRNA does not lead to a detectable protein. By contrast, the N-terminal region of CstF77.S without the intronic sequence can be readily expressed in the cell when tagged with EGFP.

### Intronic pA Usage is Part of a Feedback Mechanism to Repress CstF77 Expression

Small interfering RNAs (siRNAs) that target CstF77.L mRNA were used to examine expression of both CstF77.L and CstF77.S mRNAs. CstF77.L mRNA level significantly decreased 8 hrs after siRNA transfection and its protein level started to decrease after 16 hrs. The CstF77.S mRNA level also gradually decreased after 16 hrs of siRNA transfection. This result indicates that the expression of CstF77.S can be controlled by the CstF77.L level. By contrast, knockdown of CstF77.S mRNA did not affect the level of CstF77.L mRNA, consistent with our finding that CstF77.S mRNA does not produce any detectable protein and hence is unlikely to have any functional role. The effect of knockdown of CstF77.L or CstF77.S on the intronic pA usage using the reporter construct pRinG-77Sin-831 was examined. Knockdown of CstF-77.L expression inhibited intronic pA usage, whereas knockdown of Cstf77.S had no effect.

To further explore the autoregulatory mechanism, CstF77 protein was overexpressed in the cell. Expression of exogenous CstF77 led to increased expression of endogenous CstF77.S mRNA and decreased expression of endogenous CstF77.L mRNA. In addition, expression of exogenous CstF77 enhanced intronic pA usage for the pRinG-77Sin-831 vector. The data indicated that intronic pA usage is responsive to CstF77 expression, suggesting a negative feedback autoregulation.

Intronic polyA Usage is Controlled by the Splicing Activity

Given the significant impact of intron size and 5′SS strength on intronic polyA of CstF77, the change of splicing activity may also regulate CstF77 expression. An oligonucleotide which mimics the consensus sequence of 5′SS (Nat Biotechnol 27: 257-263 (2009)) was used, termed U1 domain (U1D) oligo. U1D can sequester U1 snRNP in the cell, thereby inhibiting 5′SS recognition by U1 snRNP. Upon treatment of U1D, the ratio of CstF77.S to CstF77.L increased by 2-fold, suggesting activation of intronic polyA. In addition, reporter assays using pRinG-77Sin-1690 showed an even more dramatic increase of intronic pA usage by ˜9-fold. To determine the role of U1 snRNP in intronic polyA of CstF77, U1-70K was knocked down, one of the components of U1 snRNP. Knocking down U1-70K (˜70% decrease at the protein level, 48 hs after siRNA transfection) led to over 50% increase of the CstF77.S/CstF77.L ratio.

The effect of U2 snRNP on intronic polyA of CstF77 was examined. siRNAs were used against SF3B1, a key component of U2 snRNP and U2AF65, a factor involved in recognition of 3′SS and recruitment of U2 snRNP. When SF3B1 was knocked down (60% decrease at the protein level, 48 hrs after siRNA transfection), the CstF77.S/CstF77.L ratio is slightly up-regulated by ˜20%. By contrast, when U2AF65 was knocked down (70% decrease at the protein level, 48 hrs after siRNA transfection), the CstF77.S/CstF77.L ratio actually decreased by ˜20%. These results indicate regulation of splicing activity can impact intronic pA usage of CstF77, and regulation of 5′SS recognition appears to be most potent.

Intronic polyA of CstF77 is Regulated During Cell Differentiation.

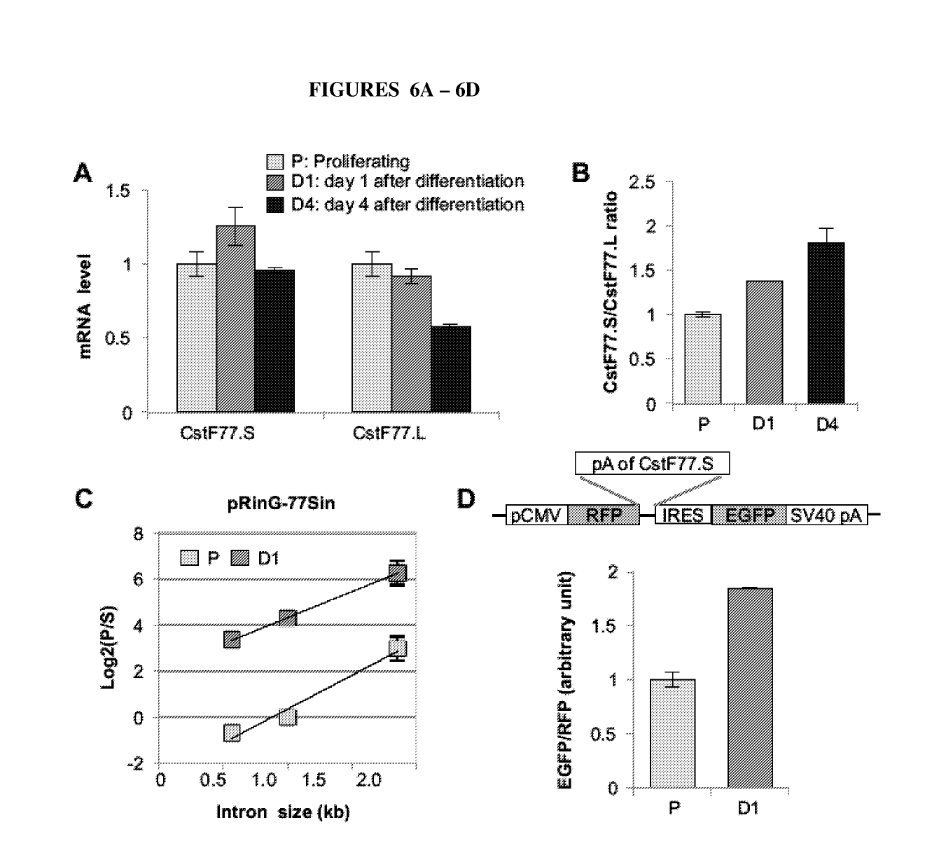

Differentiation of C2C12 myoblast cells was used as a model, in which widespread alternative splicing and APA events take place. PolyA factors are generally downregulated during C2C12 differentiation, suggesting weakening of 3′ end processing activity. The expression of CstF77.S and CstF77.L in proliferating cells and 1 day and 4 days after differentiation. CstF77.S showed increased expression by ˜20% after 1 day of differentiation but no significant change of expression after 4 days. By contrast, the expression of CstF77.L gradually decreased (FIG. 6a). The CstF77.S/CstF77.L ratio gradually increased in differentiation (FIG. 6b). This result indicates that the intronic polyA of CstF77 is upregulated in differentiation. Reporter assays using pRinG-77Sin vectors with different intron sizes were carried out, and it was determined that intronic pA usage was indeed increased in differentiated cells (FIG. 6c). To examine if this is due to increase of polyA activity per se, pRiG-77.AE was usedwhich has the pA of CstF77.S followed by SV40 pA. Unlike pRinG constructs, pRiG-77.AE plasmid does not have an intron and can be used to examine the efficiency of 3′ end processing at the CstF77.S pA without the influence of splicing. As shown in FIG. 6d, the pA usage of CsfF77.S was actually decreased in differentiation (FIG. 6d), indicating that the upregulated usage of intronic pA in differentiation is due to decreased competition of splicing with polyA.

Intronic polyA of CstF77 Globally Correlates with Regulation of 3′UTR Length Across Human and Mouse Tissues

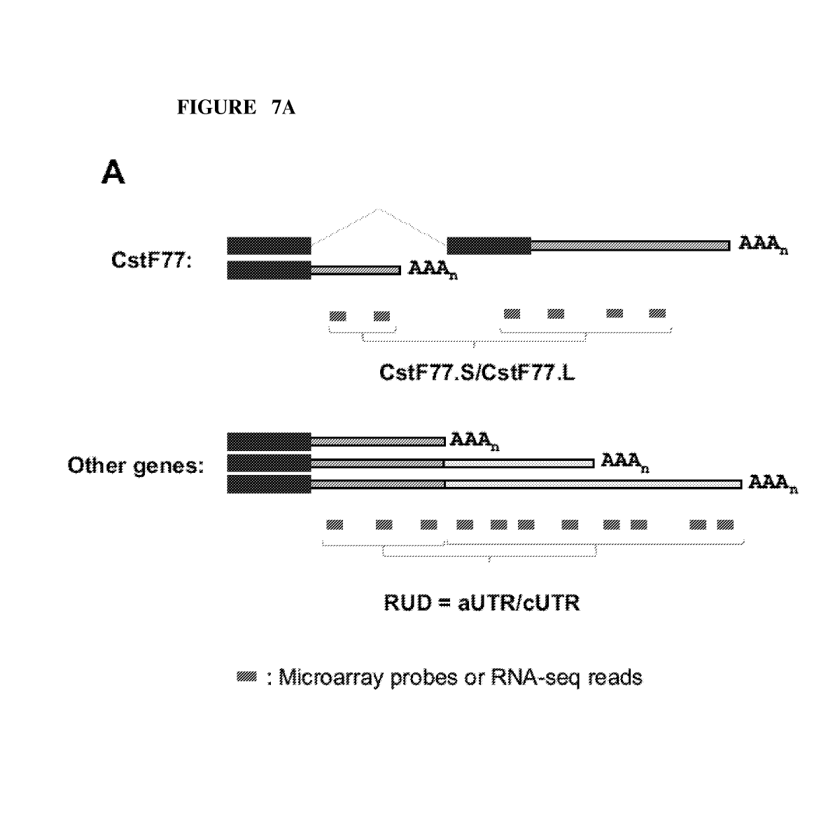

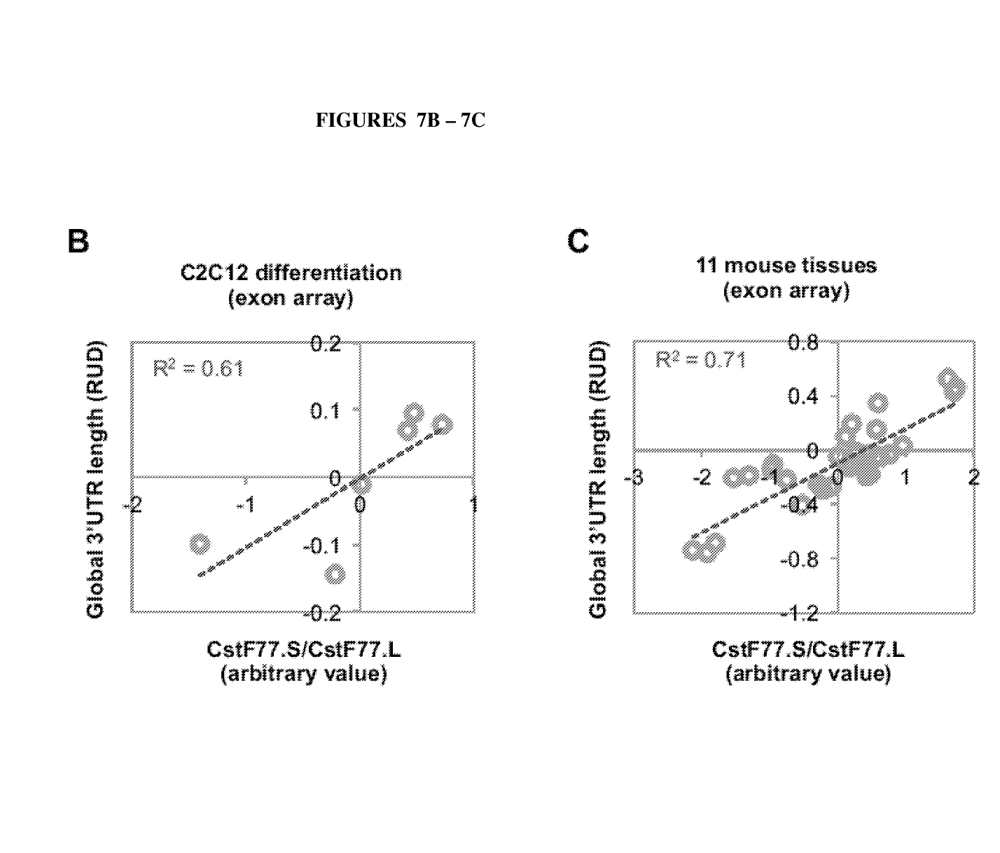

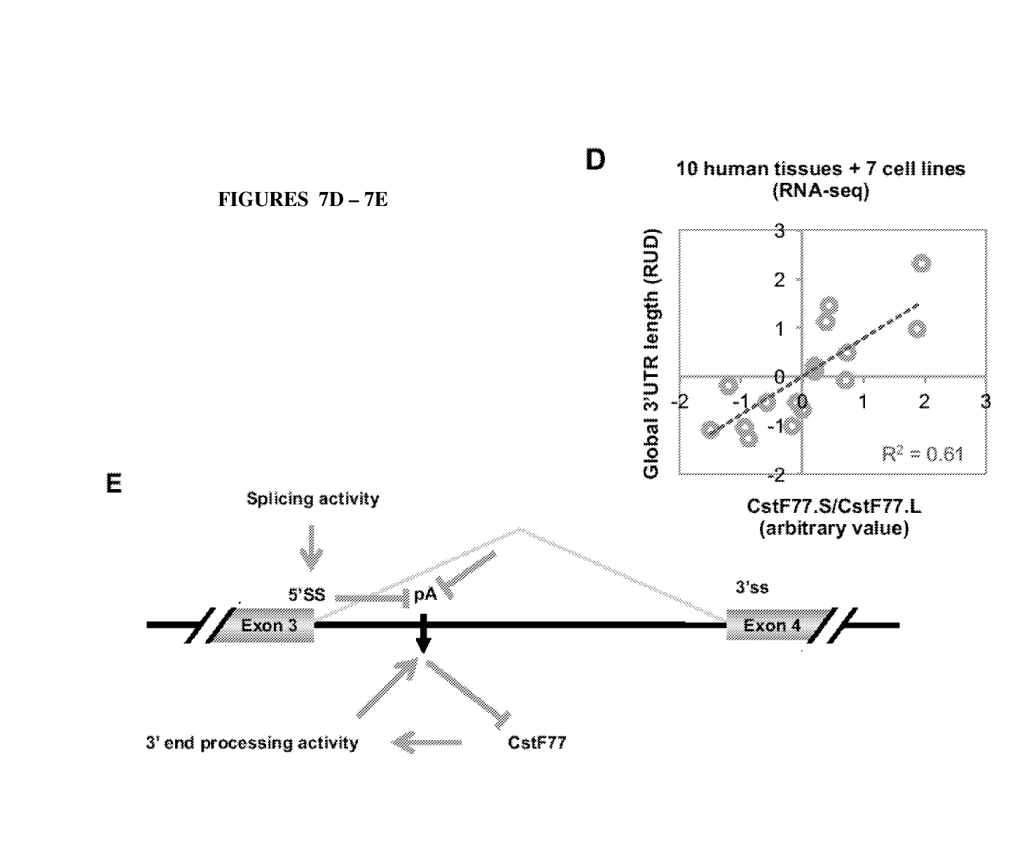

As illustrated in FIG. 7a, the CstF77.S/CstF77.L ratio was calculated by comparing the intensity of microarray probes or density of RNA-seq reads for CstF77.S with that for CstF77.L; and the global 3′UTR length was calculated by comparing the intensity of microarray probes or density of RNA-seq reads for the region upstream of the first pA in 3′UTR (called constitutive 3′UTR or cUTR) with that for the downstream region (called alternative 3′UTR or aUTR). The latter value was also called RUD. General lengthening of 3′UTR occurs during cell differentiation in C2C12 myoblast cells (Proc Natl Acad Sci USA 106: 7028-7033 (2009)). As shown in FIG. 7b, a linear correlation (R²=0.61) between CstF77.S/CstF77.L and RUD can be discerned. To examine this correlation more systematically, an exon array dataset for 11 mouse tissues and a deep sequencing dataset for 10 human tissues and 7 human cell lines were analyzed (FIG. 7b-d). Consistent with the C2C12 data, the CstF77.S/CstF77.L ratio generally correlates with the global length of 3′UTR in both human and mouse cells/tissues. This result indicates that regulation of the intronic pA of CstF77 is directly relevant to global 3′UTR length control. FIG. 7(e) shows a model for regulation of intronic polyA of CstF77 by 3′ end processing and splicing activities.

All references cited herein are incorporated herein in their entireties.

1. An oligonucleotide comprising at least one nucleic acid and an affinity moiety, wherein said nucleic acid is 30-60 nucleotides in length and said nucleic acid comprises 1-25 uracil and 5-50 thymine nucleotides.

2. The oligonucleotide of claim 1, wherein said nucleic acid comprises 3′-U₅T₄₅-5′ or 3′-U₁₅T₃₅-5′.

3. The oligonucleotide of claim 1, wherein said uracil or thymine nucleotides are contiguous.

4. (canceled)

5. The oligonucleotide of claim 1, wherein said affinity moiety is biotin.

6. The oligonucleotide of claim 1 wherein more than one nucleic acid is conjugated to said affinity moiety.

7. The oligonucleotide of claim 1 wherein the nucleic acid comprises nucleotides consisting of uracil and thymine.

8. The oligonucleotide of claim 1 wherein the affinity moiety is bound to a solid support.

9. The oligonucleotide of claim 8 wherein the affinity moiety is biotin and the solid support is a streptavidin coated bead.

10. A method to isolate nucleic acids wherein said method is capable of separating at least one nucleic acid containing a long poly (A) sequence from at least one nucleic acid containing a short poly (A) sequence, said method comprising:
a. obtaining a sample of nucleic acids containing poly (A) sequences;
b. fragmenting said nucleic acids solution to provide a solution of fragmented nucleic acids;
c. reacting said solution of fragmented nucleic acids with the oligonucleotide of claim 1 to provide a solution of nucleic acids annealed to the oligonucleotide and nucleic acids that are not annealed to the oligonucleotide;
d. removing nucleic acids having short poly (A) sequences with a stringent wash to provide a solution of nucleic acids having long poly (A) sequences annealed to the oligonucleotide;
e. contacting said solution of nucleic acids annealed to said oligonucleotide with an enzyme, wherein said enzyme releases nucleic acids from said oligonucleotide; and
f. separating said released nucleic acids to provide a solution of isolated nucleic acids.

11. The method of claim 10, wherein the sample of nucleic acids is from a cell, tissue or a subject.

12. The method of claim 10, wherein said sample of nucleic acids with a poly (A) sequence is obtained using a oligo-dT column.

13. The method of claim 10, wherein said enzyme is RNaseH.

14. A method to detect polyadenylation sites in a gene comprising:
a. obtaining a solution of nucleic acids containing poly(A) sequences;
b. fragmenting said nucleic acids to provide a solution of fragmented nucleic acids;
c. reacting said solution of fragmented nucleic acids with the oligonucleotide of claim 1 to provide a solution of nucleic acids annealed to the oligonucleotide and nucleic acids that are not annealed to the oligonucleotide;
d. removing nucleic acids having short poly (A) sequences with a stringent wash to provide a solution of nucleic acids having long poly (A) sequences annealed to the oligonucleotide;
e. contacting said solution of nucleic acids annealed to said oligonucleotide with an enzyme, wherein said enzyme releases nucleic acids from said oligonucleotide;
f. separating said released nucleic acids to provide a solution of isolated nucleic acids;
g. contacting said solution of purified nucleic acids with a kinase to provide a solution of 5′ phosphorylated nucleic acids;
h. contacting said solution of 5′ phosphorylated nucleic acids with a 3′ adapter, a 5′ adapter, and ligases suitable for ligating said adapters to the 3′ and 5′ ends of the nucleic acids to provide a solution of ligated nucleic acids;
i. contacting said solution with a reverse transcriptase to provide cDNA corresponding to said ligated nucleic acids;
j. amplifying said cDNA corresponding to said ligated nucleic acids by polymerase chain reaction to provide amplified nucleic acids;
k. sequencing said amplified nucleic acids;
l. comparing the sequences of said nucleic acids to the sequence of a reference gene; and
m. determining polyadenylation sites in the gene.

15. The method of claim 14, further comprising recording in a computer-readable form detection data indicative of detection of poly (A) sites in a gene.

16. The method of claim 14, wherein said at least one nucleic acid containing a long poly (A) sequence has more than 16 contiguous adenine nucleotides.

17. The method of claim 14, wherein said fragmenting said nucleic acids step comprises fragmenting said nucleic acids with a metal base or a metal ion solution or RNase III, or a combination thereof.

18. A method to determine the differentiation state or proliferation state of a cell comprising:
a. identifying alternative polyadenylation mRNA isoforms of CstF77 from a tissue of interest;
b. determining the ratio of CstF77 short isoforms to CstF77 long isoforms in said tissue,
c. comparing the ratio of CstF77 short isoforms to CstF77 long isoforms in said cell to a standard ratio in a control sample; and
wherein if said ratio is greater than a standard ratio in a control sample the state of said cell is a differentiating cell; and
wherein if said ratio is less than a standard ratio in a control sample the state of said cell is a proliferating cell.

19. (canceled)

20. A method to measure intronic pA usage in a cell comprising:
a. isolating and measuring alternative polyadenylation mRNA isoforms of CstF-77 from a cell of interest; and
b. determining the ratio of CstF-77 short isoforms to CstF-77 long isoforms (Cst-77.S/Cst-77.L) in said cell,
wherein intronic pA usage is positively correlated with Cst-77.S/Cst-77.L.

21. A kit comprising the oligonucleotide of claim 1 in a single container or separate containers, and instructions for use in a method according to claims 10-20.

22. The kit of claim 21, further comprising at least one of a metal base or metal ion solution, RNAse III, a wash buffer, RNAse H, a kinase, a ligase, and a reverse transcriptase.

23. The kit of claim 21 further comprising reagents for polymerase chain reaction.

24. A kit comprising a first affinity moiety that binds specifically to a CstF77 short isoform and a second affinity moiety that binds specifically to a CstF77 long isoform in separate containers, and instructions for use in a method according to claim 18.

25. The kit of claim 21 wherein said first and second affinity moiety is selected from the group consisting of linkers, biotin, nucleic acids, peptides, and antibodies and fragments thereof.

26. A computer program product comprising:
a. a computer-readable storage medium; and
b. instructions stored on the computer-readable storage medium that when executed by a computer cause the computer to:
receive poly (A) site data according to claim 14; and perform at least one of:
(i) mapping poly (A) site data to a genome;
(ii) comparing the poly (A) site data in the nucleic acid with a reference nucleic acid; and
(iii) identifying a biological marker from the poly (A) site data.

27. A computer program product according to claim 26, wherein the instructions when executed by the computer further cause the computer to search for a phenotype designation associated with the identified reference nucleic acid.

28. The method of claim 10, wherein said at least one nucleic acid containing a long poly (A) sequence has more than 16 contiguous adenine nucleotides.

29. The method of claim 10, wherein said fragmenting said nucleic acids step comprises fragmenting said nucleic acids with a metal base or a metal ion solution or RNase III, or a combination thereof.
